(19) United States
(12) Patent Application Publication
(10) Pub. No.: US 2025/0345292 A1
(43) Pub. Date: Nov. 13, 2025

(54) COMBINATION OF ALBUTEROL AND BUDESONIDE FOR THE TREATMENT OF ASTHMA

(71) Applicant: ASTRAZENECA AB, Sodertalje (SE)

(72) Inventors: Christy CAPPELLETTI, Durham, NC (US); Eva JOHNSSON, Molndal (SE); Mark COOPER, Cambridge (GB); Benjamin FENBY; Andrea MAES, Wilmington, DE (US); Laurence REILLY, St. Peter Port (GG); Robert REES, St. Peter Port (GG); Martin RIMMER, St. Peter Port (GG)

(21) Appl. No.: 18/860,366

(22) PCT Filed: Apr. 27, 2023
(86) PCT No.: PCT/US2023/020200

§ 371 Date: Oct. 25, 2024

Related U.S. Application Data
(60) Provisional application No. 63/363,767, filed on Apr. 28, 2022.

(51) Int. Cl. A61K 31/137; A61K 9/00; A61K 31/58; A61K 47/06; A61P 11/06

(52) U.S. Cl. CPC A61K 31/137; A61K 9/008; A61K 31/58; A61K 47/06; A61P 11/06

(57) ABSTRACT

The present disclosure provides a method of treating asthma in a subject at risk of asthma exacerbation, and a method of as-needed treatment or prevention of bronchoconstriction in a subject, and/or prevention of exacerbation in a subject with asthma, comprising administering as needed to the subject a composition comprising therapeutically effective amounts of albuterol and budesonide, wherein the composition is administered via a metered dose inhaler, wherein the method reduces risk of severe asthma exacerbation by at least about 15% as compared to administration of a composition comprising a same dose of albuterol alone, as measured by time-to-first severe asthma exacerbation. Also provided is a pharmaceutical composition comprising: albuterol and budesonide in the form of particles; a suspension medium comprising a hydrofluoroolefins (HFO) propellant and/or a hydrofluorocarbon propellant (HFC), and a plurality of respirable suspending particles, wherein the particles of albuterol and budesonide associate with the plurality of respirable suspending particles.

### RELATED APPLICATION

The present application claims benefit of priority to U.S. Provisional Application No. 63/363,767, filed Apr. 28, 2022, the content of which is hereby incorporated by reference in its entirety for all purposes.

### FIELD

The present disclosure provides a method of treating asthma in a subject at risk of asthma exacerbation, and a method of as-needed treatment or prevention of bronchoconstriction in a subject, and/or prevention of exacerbation in a subject with asthma, comprising administering as needed to the subject a composition comprising therapeutically effective amounts of albuterol and budesonide, wherein the composition is administered via a metered dose inhaler, wherein the method reduces risk of severe asthma exacerbation by at least about 15% as compared to administration of a composition comprising a same dose of albuterol alone, as measured by time-to-first severe asthma exacerbation. Also provided is a pharmaceutical composition comprising: albuterol and budesonide in the form of particles; a suspension medium comprising a hydrofluoroolefins (HFO) propellant and/or a hydrofluorocarbon propellant (HFC), and a plurality of respirable suspending particles, wherein the particles of albuterol and budesonide associate with the plurality of respirable suspending particles.

### BACKGROUND

Asthma is a heterogeneous disease that manifests as variable airflow obstruction with recurring symptoms driven by underlying persistent, yet fluctuating, airway inflammation. With loss of asthma control, patients often focus on obtaining immediate symptom relief by relying on their rescue medication, typically a short-acting β2-agonist (SABA). However, SABAs have little effect on underlying airway inflammation, and SABA overreliance serves as a metric for poor asthma control associated with severe exacerbation risk. Because severe exacerbations contribute to significant morbidity and mortality, exacerbation prevention represents an imperative in asthma management.

### SUMMARY

In some embodiments, the present disclosure provides a method of treating asthma in a subject at risk of asthma exacerbation, comprising administering as needed to the subject a composition comprising therapeutically effective amounts of albuterol and budesonide, wherein the composition is administered via a metered dose inhaler, wherein the method reduces risk of severe asthma exacerbation by at least about 15% as compared to administration of a composition comprising a same dose of albuterol alone, as measured by time-to-first severe asthma exacerbation.

In some embodiments, the method reduces risk of severe asthma exacerbation by at least about 20% as compared to administration of a composition comprising a same dose of albuterol alone, as measured by time-to-first severe asthma exacerbation. In some embodiments, the method reduces risk of severe asthma exacerbation by at least about 25% as compared to administration of a composition comprising a same dose of albuterol alone, as measured by time-to-first severe asthma exacerbation.

In some embodiments, a hazard ratio of the method as compared to administration of a composition comprising a same dose of albuterol alone is less than about 0.9. In some embodiments, a hazard ratio of the method as compared to administration of a composition comprising a same dose of albuterol alone is less than about 0.8. In some embodiments, a hazard ratio of the method as compared to administration of a composition comprising a same dose of albuterol alone is less than about 0.7.

In some embodiments, an annualized severe asthma exacerbation rate of the method is at least about 20% lower as compared to administration of a composition comprising a same dose of albuterol alone. In some embodiments, an annualized severe asthma exacerbation rate of the method is at least about 25% lower as compared to administration of a composition comprising a same dose of albuterol alone.

In some embodiments, the method reduces the subject's number of severe asthma exacerbations requiring hospitalization by at least 30% as compared to administration of a composition comprising a same dose of albuterol alone. In some embodiments, the method reduces the subject's number of severe asthma exacerbations requiring emergency and/or urgent care by at least 20% as compared to administration of a composition comprising a same dose of albuterol alone. In some embodiments, the method reduces the subject's annualized total systemic corticosteroid dose by at least 30% as compared to administration of a composition comprising a same dose of albuterol alone.

In some embodiments, the subject's forced expiratory volume in 1 second area under the curve from 0-6 hours (FEV₁ AUC₀₋₆) at 12 weeks following administration of the composition is greater than 110 mL. In some embodiments, the subject's FEV₁ AUC₀₋₆ at 12 weeks following administration of the composition is at least 10% higher as compared to a subject administered with a same dose of albuterol alone or a same dose of budesonide alone.

In some embodiments, the subject's trough FEV₁ at 12 weeks following administration of the composition is greater than 90 mL. In some embodiments, the subject's trough FEV₁ at 12 weeks following administration of the composition is at least 10% higher as compared to a subject administered with a composition comprising a same dose of albuterol alone or a same dose of budesonide alone.

In some embodiments, the administering comprises delivering one or more metered doses from the metered dose inhaler, wherein about 100 to about 300 μg albuterol and about 25 to about 250 μg budesonide are delivered. In some embodiments, about 120 to about 250 μg albuterol is delivered. In some embodiments, about 150 to about 200 μg albuterol is delivered. In some embodiments, about 180 μg albuterol is delivered. In some embodiments, about 50 to about 200 μg budesonide is delivered. In some embodiments, about 75 to about 175 μg budesonide is delivered. In some embodiments, about 80 or about 160 μg budesonide is delivered. In some embodiments, about 150 to about 200 μg albuterol and about 75 to about 175 μg budesonide are delivered. In some embodiments, about 180 μg albuterol and about 80 or about 160 μg budesonide are delivered.

In some embodiments, the administering comprises delivering two metered doses from the metered dose inhaler, wherein each delivered dose comprises about 60 to about 125 μg albuterol and about 25 to about 100 μg budesonide. In some embodiments, each delivered dose comprises about 90 μg albuterol and about 40 or about 80 μg budesonide. In some embodiments, the albuterol and the budesonide are in the composition at weight a ratio of about 1:1 to about 3:1. In some embodiments, the albuterol and the budesonide are in the composition at weight a ratio of about 1.1:1 to about 2.5:1. In some embodiments, the albuterol and the budesonide are in the composition at weight a ratio of about 1.125:1 or about 2.25:1.

In some embodiments, the albuterol and the budesonide are in the form of particles, wherein at least 90% of the particles by volume comprise an optical diameter of about 1 μm to about 7 μm. In some embodiments, the particles of albuterol comprise crystalline or micronized albuterol sulfate and the particles of budesonide comprise micronized budesonide. In some embodiments, the composition comprises a plurality of albuterol particles; a plurality of albuterol particles; a plurality of respirable suspending particles; and a suspension medium, wherein the plurality of albuterol particles, the plurality of budesonide particles, and the plurality of respirable suspending particles are co-suspended in the suspension medium to form a co-suspension. In some embodiments, the suspension medium is a pharmaceutically acceptable propellant selected from a hydrofluoroalkane (HFA) propellant, a hydrofluoroolefins (HFO) propellant, a hydrofluorocarbon propellant (HFC), and combination thereof. In some embodiments, the suspension medium is a propellant of pharmaceutical grade (1E)-1,3,3,3-Tetrafluoro-1-propene (HFO-1234ze(E)).

In some embodiments, the respirable suspending particles comprise a volume median optical diameter of about 0.2 μm to about 50 μm. In some embodiments, the respirable suspending particles are about 1 mg/mL to about 30 mg/mL in the suspension medium. In some embodiments, the respirable suspending particles comprise a phospholipid. In some embodiments, the phospholipid is 1,2-Distearoyl-sn-glycero-3-phosphocholine (DSPC). In some embodiments, a weight ratio of total mass of the respirable suspending particles to total mass of the albuterol and budesonide particles is about 1.5:1 to about 200:1.

In some embodiments, the present disclosure provides a method of as-needed treatment or prevention of bronchoconstriction in a subject, and/or prevention of exacerbation in a subject with asthma, comprising administering as needed to the subject a composition comprising therapeutically effective amounts of albuterol and budesonide, wherein the composition is administered via a metered dose inhaler, wherein the method reduces risk of severe asthma exacerbation by at least about 15% as compared to administration of a composition comprising a same dose of albuterol alone, as measured by time-to-first severe asthma exacerbation.

In some embodiments, the subject is a human subject 12 years of age or older. In some embodiments, the administering comprises delivering to the subject two metered doses from the metered dose inhaler, wherein about 180 μg albuterol and about 160 μg budesonide are delivered. In some embodiments, the subject is a human subject from 4 years to 11 years of age. In some embodiments, the administering comprises delivering to the subject two metered doses from the metered dose inhaler, wherein about 180 μg albuterol and about 80 μg budesonide are delivered. In some embodiments, the subject is not administered greater than 6 metered doses in a 24 hour period.

In some embodiments, the disclosure provides a pharmaceutical composition deliverable from a metered dose inhaler comprising therapeutically effective amounts of albuterol and budesonide for use in treatment of asthma in a subject at risk of asthma exacerbation, wherein the treatment reduces risk of severe asthma exacerbation by at least about 15% as compared to administration of a composition comprising a same dose of albuterol alone, as measured by time-to-first severe asthma exacerbation.

In some embodiments, the disclosure provides a pharmaceutical composition deliverable from a metered dose inhaler comprising therapeutically effective amounts of albuterol and budesonide for use in as-needed treatment or prevention of bronchoconstriction in a subject and/or prevention of exacerbation in a subject with asthma, wherein the method reduces risk of severe asthma exacerbation by at least about 15% as compared to administration of a composition comprising a same dose of albuterol alone, as measured by time-to-first severe asthma exacerbation.

In some embodiments, the pharmaceutical composition comprises a plurality of albuterol particles; a plurality of albuterol particles; a plurality of respirable suspending particles; and a suspension medium, wherein the plurality of albuterol particles, the plurality of budesonide particles, and the plurality of respirable suspending particles are co-suspended in the suspension medium to form a co-suspension. In some embodiments, the suspension medium is a propellant of pharmaceutical grade (1E)-1,3,3,3-Tetrafluoro-1-propene (HFO-1234ze(E)). In some embodiments, the respirable suspending particles comprise a phospholipid. In some embodiments, the phospholipid is 1,2-Distearoyl-sn-glycero-3-phosphocholine (DSPC).

In some embodiments, a hazard ratio of the treatment as compared to administration of a composition comprising a same dose of albuterol alone is less than about 0.9. In some embodiments, an annualized severe asthma exacerbation rate of the treatment is at least about 20% lower as compared to administration of a composition comprising a same dose of albuterol alone.

### BRIEF DESCRIPTION OF THE DRAWINGS

The following drawings form part of the present specification and are included to further demonstrate exemplary embodiments of certain aspects of the present disclosure.

FIG. 1A shows patient enrollment and randomization in the clinical trial study as described in Example 1. Safety and full analysis sets are indicated in bold typeface. Abbreviations: EOS, end of study.

FIG. 1B shows a table of patient demographics and clinical characteristics at screening for the clinical trial study as described in Example 1. Abbreviations: BMI, body mass index; FEV₁, forced expiratory volume in the first second; FVC, forced vital capacity; LABA, long-acting β2-agonist; LAMA, long-acting muscarinic antagonist; LTRA, leukotriene receptor antagonist; SD, standard deviation.

FIG. 2 shows time-to-first severe exacerbation data (efficacy estimand) as determined during the randomized treatment period of the clinical trial study as described in Example 1. Abbreviations: CI, confidence interval; HR, hazard ratio.

FIG. 3 shows a table of secondary endpoint results (efficacy estimand) in the overall population of the clinical study trial as described in Example 1. Abbreviations: ACQ-5, Asthma Control Questionnaire-5; AQLQ+12, Asthma Quality of Life Questionnaire; CI, confidence interval; SD, standard deviation.

### DETAILED DESCRIPTION OF THE EMBODIMENTS

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y and any numbers that fall within x and y.

As used herein, the term “active agent” includes any agent, drug, compound, composition, or other substance that may be used on, or administered to a subject, e.g., a human or animal subject, for any purpose, including therapeutic, pharmaceutical, pharmacological, diagnostic, cosmetic, and prophylactic agents and immunomodulators. The term “active agent” may be used interchangeably with the terms “drug,” “pharmaceutical,” “medicament,” “drug substance,” and “therapeutic.” In some embodiments, the active agent of the present disclosure comprises albuterol and/or budesonide.

As used herein, the terms “associate,” “associate with,” and “association” refer to an interaction or relationship between a chemical entity, composition, or structure by proximity to a surface, such as the surface of another chemical entity, composition, or structure. The association includes, for example, adsorption, adhesion, hydrogen bonding, ionic bonding and electrostatic attraction, Lifshitz-van der Waals interactions and polar interactions. As described herein, active agent particles may associate with suspending particles to form a co-suspension, where there is substantially no visible separation between the suspending particles and the active agent particles or flocculates thereof due to differences in buoyancy within a propellant.

As used herein, the term “respirable” refers to particles, aggregates, drops, etc. sized such that they can be inhaled and reach the airways of the lung.

As used herein, “suspending particles” refer to a material or combination of materials that is acceptable for respiratory delivery, and acts as a vehicle for active agent particles. Suspending particles interact with the active agent particles to facilitate repeatable dosing, delivery or transport of active agent to the target site of delivery, e.g., the respiratory tract (i.e., “respirable suspending particles”). In some embodiments, the suspending particles described herein are dispersed within a suspension medium including a propellant or propellant system, and can be configured according to any shape, size or surface characteristic suited to achieving a desired suspension stability or active agent delivery performance. Exemplary suspending particles include particles that exhibit a particle size that facilitates respiratory delivery of active agent and have physical configurations suited to formulation and delivery of the stabilized suspensions as described herein.

As used herein, “mass mean aerodynamic diameter” or “MMAD” refers to the aerodynamic diameter of an aerosol below which 50% of the mass of the aerosol consists of particles with an aerodynamic diameter smaller than the MMAD, with the MMAD being calculated according to monograph 601 of the United States Pharmacopeia (USP).

As used herein, “optical diameter” refers to the size of a particle as measured by the Fraunhofer diffraction mode using a laser diffraction particle size analyzer equipped with a dry powder dispenser (e.g., Sympatec GmbH, Clausthal-Zellerfeld, Germany).

As used herein, the term “suspension medium” refers to a substance providing a continuous phase within which active agent particles and suspending particles can be dispersed to provide a co-suspension formulation. In some embodiments, the suspension medium used in co-suspension formulations described herein includes a propellant. As used herein, the term “propellant” refers to one or more pharmacologically inert substances which exert a sufficiently high vapor pressure at normal room temperature to propel a medicament from the canister of a metered dose inhalers (MDI) to a subject on actuation of the MDI's metering valve. The term “propellant” refers to both a single propellant and to a combination of two or more different propellants forming a “propellant system.”

As used herein, the term “co-suspension” refers to a suspension of two or more types of particles having different compositions within a suspension medium, wherein one type of particle associates at least partially with one or more of the other particle types. The association leads to an observable change in one or more characteristics of at least one of the individual particle types suspended in the suspension medium. Characteristics modified by the association may include, for example, one or more of the rate of aggregation or flocculation, the rate and nature of separation, i.e. sedimentation or creaming, density of a cream or sediment layer, adhesion to container walls, adhesion to valve components, and rate and the level of dispersion upon agitation. Exemplary methods for assessing whether a co-suspension is present are known to one of ordinary skill in the art, e.g., as described in WO 2010/138862.

A “therapeutically effective amount” is the amount of compound, e.g., albuterol and/or budesonide as described herein, which achieves a therapeutic effect by inhibiting a condition or disorder in a patient, e.g., asthma and/or bronchoconstriction, or by prophylactically inhibiting or preventing the onset of a condition or disorder. A therapeutically effective amount may be an amount which relieves to some extent one or more symptoms of a condition or disorder in a patient; returns to normal either partially or completely one or more physiological or biochemical parameters associated with or causative of the condition or disorder; and/or reduces the likelihood of the onset of the condition of disorder.

### Treatment of Asthma

In some embodiments, the present disclosure provides compositions and methods for treatment of asthma, including as-needed treatment or prevention of bronchoconstriction and prevention of asthma exacerbation.

Asthma is a chronic respiratory condition that causes inflammation and narrowing of the airways. As used herein, “asthma” refers to asthma of any type and genesis, including intrinsic (non-allergic) asthma and extrinsic (allergic) asthma, mild asthma, moderate asthma, severe asthma, bronchitic asthma, exercise-induced asthma, occupational asthma, and asthma induced following bacterial and/or viral infection. As used herein, “asthma” also include wheezy-infant syndrome.

Asthma may be diagnosed as “intermittent” or “persistent,” with persistent asthma further categorized as “mild,” “moderate,” and “severe.” The severity of an individual's asthma diagnosis may be determined based on a number of factors, including but not limited to the frequency and severity of past and current asthma symptoms and the individual's lung capacity, which may be measured using spirometry, peak flow measurements (i.e., the amount and rate of air that can be forced from the lungs), lung volume testing (i.e., volume of air in the lungs), diffusing capacity testing (i.e., how easily oxygen enters the bloodstream), and/or exercise testing. A spirometry test measures forced expiratory volume (FEV), e.g., the amount of air forced from the lungs in one second (FEV₁). In general, asthma diagnoses can be characterized as follows:

| | Daytime | Nighttime | |
|---|---|---|---|
| Diagnosis | symptoms | Symptoms | Lung function test |
| Intermittent | <2x/week | <2x/month | FEV₁ >80%; Peak flow rate varies <20% |
| Mild | 3-4x/week | 3-4x/month | FEV₁ >80%; Peak flow rate varies <20% |
| Moderate | Daily | >5x/month | FEV₁ 60-80%; Peak flow rate varies >30% |
| Severe | Multiple per day | Nightly | FEV₁ <60%; Peak flow rate varies >30% |

An asthma exacerbation, also known as an asthma attack or acute, refers to a swelling and inflammation of the airways, i.e., bronchoconstriction, which leads to progressive increase in asthma symptoms, including coughing, shortness of breath, wheezing, chest tightness, increased respiratory rate, increased pulse rate, and/or decreased lung function. As used herein, “severe exacerbation” or “severe asthma exacerbation” refers to any deterioration of asthma that leads to at least one of the following conditions: (i) ≥3 consecutive days' treatment with systemic glucocorticoids (SCS) to treat worsening symptoms of asthma; a single depot injection was considered equivalent to a 3-day burst; (ii) an emergency room or urgent care visit (defined as evaluation and treatment for <24 hours in an emergency department or urgent care center) due to asthma that require SCS as above; and (iii) an in-patient hospitalization (defined as admission to an in-patient facility and/or evaluation of treatment in a healthcare facility for ≥24 hours) due to asthma.

In some embodiments, the disclosure provides a method of treating asthma in a subject at risk of asthma exacerbation, comprising administering as needed to the subject a composition comprising therapeutically effective amounts of albuterol and budesonide, wherein the composition is administered via a metered dose inhaler, wherein the method reduces risk of severe asthma exacerbation by at least about 15% as compared to administration of a same dose of albuterol alone, as measured by time-to-first severe asthma exacerbation. In some embodiments, the disclosure provides a pharmaceutical composition deliverable from a metered dose inhaler comprising therapeutically effective amounts of albuterol and budesonide for use in treatment of asthma in a subject at risk of asthma exacerbation, wherein the treatment reduces risk of severe asthma exacerbation by at least about 15% as compared to administration of a composition comprising a same dose of albuterol alone, as measured by time-to-first severe asthma exacerbation.

In some embodiments, the disclosure provides a method of as-needed treatment or prevention of bronchoconstriction in a subject, and/or prevention of exacerbation in a subject with asthma, comprising administering as needed to the subject a composition comprising therapeutically effective amounts of albuterol and budesonide, wherein the composition is administered via a metered dose inhaler, wherein the method reduces risk of severe asthma exacerbation by at least about 15% as compared to administration of a same dose of albuterol alone, as measured by time-to-first severe asthma exacerbation. In some embodiments, the disclosure provides a pharmaceutical composition deliverable from a metered dose inhaler comprising therapeutically effective amounts of albuterol and budesonide for use in as-needed treatment or prevention of bronchoconstriction in a subject and/or prevention of exacerbation in a subject with asthma, wherein the method reduces risk of severe asthma exacerbation by at least about 15% as compared to administration of a composition comprising a same dose of albuterol alone, as measured by time-to-first severe asthma exacerbation.

As used herein, “albuterol,” also known as salbutamol, refers to a compound with chemical name 4-(2-(tert-butylamino)-1-hydroxyethyl)-2-(hydroxymethyl) phenol or a pharmaceutically acceptable salt, solvate, or ester thereof. In some embodiments, the composition described herein comprises albuterol sulfate, which is a racemic salt of albuterol. One example of albuterol sulfate has the chemical name α¹-[(tert-butylamino)methyl]-4-hydroxy-m-xylene-α,α′-diol sulfate (2:1) (salt), and has the following chemical structure:

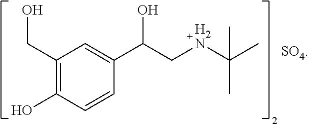

Albuterol is a short/rapid-acting β2-adrenoreceptor agonist (SABA), inducing airway smooth muscle relaxation and reducing or preventing bronchoconstriction. In clinical practice, albuterol is used as an as needed (“prn”) reliever therapy. See, e.g., Global Initiative for Asthma 2018. In general, subjects who require frequent albuterol reliever therapy are at higher risk of long-term adverse effects, including more frequent and/or more severe asthma exacerbations.

As used herein, “budesonide” refers to a corticosteroid with chemical name (RS)-11β,21-dihydroxy-16α,17α-(butylidenebis(oxy)) pregna-1,4-diene-3,20-dione. In some embodiments, the composition described herein comprises a racemic mixture of budesonide. In some embodiments, the composition described herein comprises the (22R) and (22S) epimers of budesonide. In some embodiments, budesonide has the following chemical structure (* indicating stereocenter):

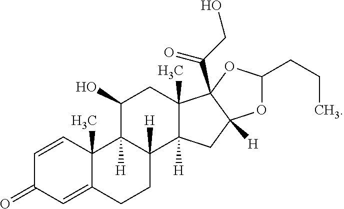

Budesonide is an anti-inflammatory corticosteroid that exhibits potent glucocorticoid and weak mineralocorticoid activity and is approved worldwide in orally inhaled formulations for treatment of asthma and chronic obstructive pulmonary disease (COPD), both as a mono-product and in combination with formoterol, a long/rapid-acting β2-agonist (LABA), as maintenance therapy for individuals with moderate to severe asthma.

It was presently discovered that simultaneous administration of a composition comprising a combination of a SABA, e.g., albuterol, with an inhaled corticosteroid (ICS), e.g., budesonide, when asthma exacerbation symptoms occur (i.e., as-needed administration) provided subjects with both symptom relief and treatment of persistent airway inflammation, without significantly increasing their overall steroid load. It was further discovered that when subjects use the combination of albuterol and budesonide for as-needed treatment of asthma exacerbation and/or as-needed treatment or prevention of bronchoconstriction, their risk of experiencing further asthma exacerbations was lower than subjects using albuterol alone.

In some embodiments, the subject is diagnosed with mild asthma. In some embodiments, the subject is diagnosed with moderate asthma. In some embodiments, the subject is diagnosed with severe asthma. In some embodiments, the subject exhibits symptoms associated with mild asthma. In some embodiments, the subject exhibits symptoms associated with moderate asthma. In some embodiments, the subject exhibits symptoms associated with severe asthma. In some embodiments, the subject is a human subject. In some embodiments, the subject is a human subject 4 years of age or older. In some embodiments, the subject is a human subject about 4 years to about 11 years of age. In some embodiments, the subject is a human subject about 12 years of age or older. In some embodiments, the subject is a human subject about 12 years to about 17 years of age or older. In some embodiments, the subject is a human subject about 18 years of age or older. It will be understood by one of ordinary skill in the art that “X years of age” for a subject includes subjects from the day that the subject turns X years old through the day that the subject turns X+1 years old.

Throughout the present application, when comparing the efficacy (e.g., as measured by time-to-first severe asthma exacerbation, hazard ratio, annualized severe asthma exacerbation rate, number of severe asthma exacerbations, as described herein) of two or more compositions that comprise different active agents, e.g., a combination of albuterol and budesonide vs. albuterol alone, it will be understood by one of ordinary skill in the art that except for the indicated active agents, the compositions are otherwise identical or substantially identical, administered in the same or substantially the same manner and, where appropriate, at the same or substantially the same dose or dose equivalent to the subject. It will be further understood that when comparing the efficacy of two or more compositions, the comparison may be made in the same subject or multiple subjects.

As used herein, “time-to-first severe asthma exacerbation” is defined in the context of a time period in which the subject is monitored, e.g., a clinical trial study period, and is calculated as the time from which the monitoring begins, e.g., date of randomization of the clinical trial study subjects, until the start date of the first severe asthma exacerbation. One of ordinary skill in the art would understand that, when comparing efficacy of compositions described herein, the starting point for measuring time-to-first severe asthma exacerbation is selected such that the timing is comparable. For example, in a clinical trial study in which a first group of subjects is administered with a composition comprising albuterol and budesonide and a second group of subjects is administered with albuterol alone, the starting point for calculating time-to-first severe asthma exacerbation is the date of randomization of the clinical trial study subject groups, thereby providing a point of comparison for assessing time-to-first severe asthma exacerbation.

In some embodiments, a longer time-to-first severe asthma exacerbation is indicative of higher drug efficacy for asthma prevention and/or treatment. In some embodiments, a longer time-to-first severe asthma exacerbation correlates with reduced risk of severe asthma exacerbation. In some embodiments, a 15% longer time-to-first severe asthma exacerbation corresponds to 15% reduction in risk of severe asthma exacerbation. In some embodiments, a 20% longer time-to-first severe asthma exacerbation corresponds to 20% reduction in risk of severe asthma exacerbation. In some embodiments, a 25% longer time-to-first severe asthma exacerbation corresponds to 25% reduction in risk of severe asthma exacerbation.

In some embodiments, administration of the composition comprising albuterol and budesonide as described herein provides an increased time-to-first severe asthma exacerbation, and therefore reduced risk of severe asthma exacerbation, as compared to administration of a composition comprising a same dose of albuterol alone. In some embodiments, administration of the composition comprising albuterol and budesonide provides at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% increase in time-to-first severe asthma exacerbation as compared to administration of a composition comprising a same dose of albuterol alone. In some embodiments, administration of the composition comprising albuterol and budesonide reduces risk of severe asthma exacerbation by at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% as compared to administration of a composition comprising a same dose of albuterol alone.

In some embodiments, administration of the composition comprising albuterol and budesonide as described herein provides at least 15% increase in time-to-first severe asthma exacerbation, and therefore reduces risk of severe asthma exacerbation by at least 15%, as compared to administration of a composition comprising albuterol alone. In some embodiments, administration of the composition comprising albuterol and budesonide provides at least 20% increase in time-to-first severe asthma exacerbation, and therefore reduces risk of severe asthma exacerbation by at least 20%, as compared to administration of a composition comprising albuterol alone. In some embodiments, administration of the composition comprising albuterol and budesonide provides at least 25% increase in time-to-first severe asthma exacerbation, and therefore reduces risk of severe asthma exacerbation by at least 25%, as compared to administration of a composition comprising albuterol alone.

In some embodiments, administration of the composition comprising albuterol and budesonide described herein as compared to a composition comprising albuterol alone provides a hazard ratio of less than 1. Hazard ratio indicates the relative hazard of two conditions, e.g., a treatment condition (e.g., administration of albuterol and budesonide) and a control condition (e.g., administration of albuterol alone). Thus, a hazard ratio of less than 1 for a treatment vs. control condition indicates a lower hazard rate for the treatment as compared to the control. In some embodiments, the hazard ratio is determined based on time-to-first severe exacerbation, as described herein. In some embodiments, administration of the composition comprising albuterol and budesonide as compared to a composition comprising albuterol alone provides a hazard ratio of less than 1, less than about 0.95, less than about 0.9, less than about 0.85, less than about 0.8, less than about 0.75, less than about 0.7, less than about 0.65, less than about 0.6, less than about 0.55, or less than about 0.5. In some embodiments, administration of the composition comprising albuterol and budesonide as compared to a composition comprising albuterol alone provides a hazard ratio of less than about 0.9. In some embodiments, administration of the composition comprising albuterol and budesonide as compared to a composition comprising albuterol alone provides a hazard ratio of less than 0.8. In some embodiments, administration of the composition comprising albuterol and budesonide as compared to a composition comprising albuterol alone provides a hazard ratio of less than 0.7.

In some embodiments, administration of the composition comprising albuterol and budesonide as described herein provides a lower annualized severe asthma exacerbation rate as compared to administration of a composition comprising albuterol alone. As used herein, “annualized severe asthma exacerbation rate” is the average number of severe asthma exacerbations experienced by a subject in a year. In some embodiments, annualized severe asthma exacerbate rate is calculated according to the following formula:

$$Annualized severe asthma exacerbation rate = \frac{\sum number of severe exacerbations \times 365.25}{monitored treatment time period}$$

In some embodiments, the monitored treatment time period is a defined time period in which a subject is administered with the compositions described herein, e.g., a clinical trial study period as described herein.

In some embodiments, administration of the composition comprising albuterol and budesonide as described herein has at least about 10%, at least about 15%, at least about 20%, at least about 25%, at least about 30%, at least about 35%, at least about 40%, at least about 45%, or at least about 50% lower annualized severe asthma exacerbation rate as compared to administration of a composition comprising a same dose of albuterol alone. In some embodiments, administration of the composition comprising albuterol and budesonide has at least about at least about 20% lower annualized severe asthma exacerbation rate as compared to administration of a composition comprising a same dose of albuterol alone. In some embodiments, administration of the composition comprising albuterol and budesonide has at least about at least about 25% lower annualized severe asthma exacerbation rate as compared to administration of a composition comprising a same dose of albuterol alone. In some embodiments, administration of the composition comprising albuterol and budesonide has at least about at least about 30% lower annualized severe asthma exacerbation rate as compared to administration of a composition comprising a same dose of albuterol alone.

In some embodiments, administration of the composition comprising albuterol and budesonide as described herein reduces the subject's number of severe asthma exacerbations requiring hospitalization by at least about 10%, at least about 15%, at least about 20%, at least about 25%, at least about 30%, at least about 35%, at least about 40%, at least about 45%, or at least about 50% as compared to administration of a composition comprising a same dose of albuterol alone. In some embodiments, administration of the composition comprising albuterol and budesonide as described herein reduces the subject's number of severe asthma exacerbations requiring emergency and/or urgent care by at least about 10%, at least about 15%, at least about 20%, at least about 25%, at least about 30%, at least about 35%, at least about 40%, at least about 45%, or at least about 50% as compared to administration of a composition comprising a same dose of albuterol alone.

In some embodiments, administration of the composition comprising albuterol and budesonide as described herein reduces the subject's annualized total systemic corticosteroid dose by at least about 10%, at least about 15%, at least about 20%, at least about 25%, at least about 30%, at least about 35%, at least about 40%, at least about 45%, or at least about 50% as compared to administration of a composition comprising a same dose of albuterol alone. In some embodiments, the method described herein reduces the number of severe asthma exacerbations, thereby reducing the need for systemic corticosteroid treatment, e.g., methylprednisone, triamcinolone, prednisolone, and prednisone. High systemic corticosteroid exposure can have adverse effects, including loss of bone density and osteoporosis, hypertension, gastrointestinal ulcers/bleeds, slower or stunted growth in children, diabetes, immunosuppression, development of cataracts, and psychiatric disorders. See, e.g., Bleecker et al., Am J Respir Crit Care Med 201 (3), 276-293 (2020) and Price et al., Eur Repsir Rev 29, 190151 (2020).

As used herein, “annualized total systemic corticosteroid dose” is the average dose of corticosteroids, including glucocorticoids and mineralocorticoids, received by the subject in a year, e.g., to treat severe asthma exacerbation. In some embodiments, annualized total systemic corticosteroid exposure is calculated according to the following formula:

$$Annualized total systemic corticosteroid dose = \frac{\sum systemic corticosteroid dose \times 365.25}{monitored treatment time period}$$

In some embodiments, the systemic corticosteroid dose is normalized to an equipotent dose of prednisone, also referred to herein as the “prednisone equivalent.” In some embodiments, the monitored treatment time period is a defined time period in which a subject is administered with the compositions described herein, e.g., a clinical trial study period as described herein.

In some embodiments, administration of a composition comprising albuterol and budesonide as described herein improves the subject's lung function, e.g., as measured by spirometry test, peak flow measurement, lung volume testing, diffusing capacity testing, and/or exercising testing as described herein. In some embodiments, administration of a composition comprising albuterol and budesonide as described herein increases the subject's FEV₁ area under the curve from 0 to 6 hours (FEV₁ AUC₀₋₆), i.e., the FEV₁ of the subject as calculated from 0 to 6 hours post administration. In some embodiments, the subject's FEV₁ AUC₀₋₆ at 12 weeks following administration of the composition comprising albuterol and budesonide as described herein is greater than about 100 mL, greater than about 105 mL, greater than about 110 mL, greater than about 115 mL, greater than about 120 mL, greater than about 125 mL, greater than about 130 mL, greater than about 140 mL, greater than about 150 mL, greater than about 160 mL, greater than about 170 mL, greater than about 180 mL, greater than about 190 mL, or greater than about 200 mL. In some embodiments, the subject's FEV₁ AUC₀₋₆ at 12 weeks following administration of the composition comprising albuterol and budesonide as described herein is at least 10 mL, at least 15 mL, at least 20 mL, at least 25 mL, at least 30 mL, at least 35 mL, at least 40 mL, at least 45 mL, or at least 50 mL greater than the FEV₁ AUC₀₋₆ of a subject at 12 weeks following administration of albuterol alone, budesonide alone, or a placebo composition.

In some embodiments, the subject's FEV₁ AUC₀₋₆ at 12 weeks following administration of the composition comprising albuterol and budesonide as described herein is at least 10%, at least 15%, at least 20%, at least 25%, at least 30%, at least 35%, at least 40%, at least 45%, or at least 50% higher as compared to the FEV₁ AUC₀₋₆ of a subject at 12 weeks following administration of a same dose of albuterol alone. In some embodiments, the subject's FEV₁ AUC₀₋₆ at 12 weeks following administration of the composition comprising albuterol and budesonide as described herein is at least 10%, at least 15%, at least 20%, at least 25%, at least 30%, at least 35%, at least 40%, at least 45%, or at least 50% higher as compared to the FEV₁ AUC₀₋₆ of a subject at 12 weeks following administration of a same dose of budesonide alone. In some embodiments, the subject's FEV₁ AUC₀₋₆ at 12 weeks following administration of the composition comprising albuterol and budesonide as described herein is at least 15%, higher as compared to the FEV₁ AUC₀₋₆ of a subject at 12 weeks following administration of a same dose of albuterol alone or a same dose of budesonide alone.

In some embodiments, administration of a composition comprising albuterol and budesonide as described herein increases the subject's trough FEV₁, i.e., the FEV₁ of the subject prior to administration (also known as the “pre-dose FEV₁”). In some embodiments, the subject's trough FEV₁ at 12 weeks following administration of the composition comprising albuterol and budesonide as described herein is greater than about 70 mL, greater than about 75 mL, greater than about 80 mL, greater than about 85 mL, greater than about 90 mL, greater than about 95 mL, greater than about 100 mL. In some embodiments, the subject's trough FEV₁ at 12 weeks following administration of the composition comprising albuterol and budesonide as described herein is at least 5 mL, at least 10 mL, at least 15 mL, at least 20 mL, at least 25 mL, at least 30 mL, at least 35 mL, at least 40 mL, at least 45 mL, or at least 50 mL greater than the trough FEV₁ of a subject at 12 weeks following administration of albuterol alone, budesonide alone, or a placebo composition.

In some embodiments, the subject's trough FEV₁ at 12 weeks following administration of the composition comprising albuterol and budesonide as described herein is at least 5%, at least 10%, at least 15%, at least 20%, at least 25%, at least 30%, at least 35%, at least 40%, at least 45%, or at least 50% higher as compared to the trough FEV₁ of a subject at 12 weeks following administration of a same dose of albuterol alone. In some embodiments, the subject's trough FEV₁ at 12 weeks following administration of the composition comprising albuterol and budesonide as described herein is at least 5%, at least 10%, at least 15%, at least 20%, at least 25%, at least 30%, at least 35%, at least 40%, at least 45%, or at least 50% higher as compared to the trough FEV₁ of a subject at 12 weeks following administration of a same dose of budesonide alone. In some embodiments, the subject's trough FEV₁ at 12 weeks following administration of the composition comprising albuterol and budesonide as described herein is at least 10% higher as compared to the trough FEV₁ of a subject at 12 weeks following administration of a same dose of albuterol alone or a same dose of budesonide alone.

### Metered Dose Inhaler

As described in relation to the methods provided herein, the composition comprising albuterol and budesonide described herein may be used in a metered dose inhaler (MDI) system. MDIs are configured to deliver a specific amount of a medicament in aerosol form. In some embodiments, an MDI system includes a pressurized, liquid phase formulation-filled canister disposed in an actuator formed with a mouthpiece. The MDI system may include the formulations described herein, which include a suspension medium, at least one species of active agent particles and at least one species of suspending particles. The canister used in the MDI be any of any suitable configuration, and in one exemplary embodiment, the canister may have a volume ranging from about 5 mL to about 25 mL, such as, for example a canister having a 19 mL volume. After shaking the device, the mouthpiece is inserted into a subject's mouth between the lips and teeth. The subject typically exhales deeply to empty the lungs and then takes a slow deep breath while actuating the MDI cartridge.

When actuated, the specified volume of formulation travels to the expansion chamber, out the actuator nozzle and into a high-velocity spray that is drawn into the lungs of a patient. In some embodiments, the dose of active agent, i.e., albuterol and budesonide, delivered throughout emptying of an MDI canister is not more than 20% greater than the mean delivered dose and is not less than 20% less than the mean delivered dose. In some embodiments, the dose of active agent, i.e., albuterol and budesonide, delivered throughout emptying of an MDI canister is not more than 15% greater or less than the mean delivered dose. In some embodiments, the dose of active agent, i.e., albuterol and budesonide, delivered throughout emptying of an MDI canister is not more than 10% greater or less than the mean delivered dose.

Inside an exemplary cartridge is a metering valve including a metering chamber capable of holding a defined volume of the formulation (e.g., 63 μL or any other suitable volume available in commercially available metering valves), which is released into an expansion chamber at the distal end of the valve stem when actuated. The actuator retains the canister and may also include a port with an actuator nozzle for receiving the valve stem of the metering valve. When actuated, the specified volume of formulation travels to the expansion chamber, out the actuator nozzle and into a high-velocity spray that is drawn into the lungs of a subject.

Exemplary MDIs and methods of use thereof are further described, e.g., in WO 2010/138862, WO 2010/138868, and WO 2010/138884.

### Dosage Amounts

In some embodiments, the administering comprises delivering to the subject one or more metered doses from the MDI through inhalation, wherein about 100 to about 300 μg albuterol and about 25 to about 250 μg budesonide is delivered. In some embodiments, about 120 to about 250 μg albuterol and about 50 to about 200 μg budesonide is delivered. In some embodiments, about 140 to about 230 μg albuterol and about 60 to about 180 μg budesonide is delivered. In some embodiments, about 150 to about 200 μg albuterol and about 75 to about 175 μg budesonide is delivered. In some embodiments, about 170 to about 190 μg albuterol and about 80 to about 160 μg budesonide is delivered. In some embodiments, about 180 μg albuterol and about 80 μg budesonide is delivered. In some embodiments, about 180 μg albuterol and about 160 μg budesonide is delivered.

In some embodiments, the administering comprises delivering to the subject 1 metered dose in a 24 hour period via inhalation. In some embodiments, administering comprises the subject inhaling 2 metered doses in a 24 hour period. In some embodiments, administering comprises the subject inhaling 3 metered doses in a 24 hour period. In some embodiments, administering comprises the subject inhaling 4 metered doses in a 24 hour period. In some embodiments, administering comprises the subject inhaling 5 metered doses in a 24 hour period. In some embodiments, administering comprises the subject inhaling 6 metered doses in a 24 hour period. In some embodiments, no more than 6 metered doses are administered to the subject in a 24 hour period. Unless otherwise specified, each metered dose comprises one actuation by the metered dose inhaler.

In some embodiments, the administering comprises delivering one or more metered doses from the MDI to the subject through inhalation, wherein the total daily delivered dose is about 100 to about 300 μg albuterol and about 25 to about 250 μg budesonide. The term “total daily delivered dose” refers to the cumulative amount of active agent, e.g., albuterol and budesonide, delivered in a 24 hour period, independent of the number of metered doses administered. In some embodiments, the total daily delivered dose is about 200 to about 600 μg albuterol and about 50 to about 500 μg budesonide. In some embodiments, the total daily delivered dose is about 300 to about 900 μg albuterol and about 75 to about 750 μg budesonide. In some embodiments, the total daily delivered dose is about 400 to about 1200 μg albuterol and about 100 to about 1000 μg budesonide. In some embodiments, the total daily delivered dose is about 500 to about 1500 μg albuterol and about 125 to about 1250 μg budesonide. In some embodiments, the total daily delivered dose is about 600 to about 1800 μg albuterol and about 150 to about 1500 μg budesonide.

In some embodiments, the total daily delivered dose is about 170 to about 190 μg albuterol and about 80 to about 160 μg budesonide. In some embodiments, the total daily delivered dose is about 340 to about 3800 μg albuterol and about 160 to about 320 μg budesonide. In some embodiments, the total daily delivered dose is about 510 to about 570 μg albuterol and about 240 to about 480 μg budesonide. In some embodiments, the total daily delivered dose is about 680 to about 760 μg albuterol and about 320 to about 640 μg budesonide. In some embodiments, the total daily delivered dose is about 850 to about 950 μg albuterol and about 400 to about 800 μg budesonide. In some embodiments, the total daily delivered dose is about 1020 to about 1140 μg albuterol and about 480 to about 960 μg budesonide.

In some embodiments, the administering comprises delivering to the subject two metered doses via inhalation from the MDI, wherein each delivered dose comprises about 50 to about 150 μg albuterol and about 12 to about 125 μg budesonide. In some embodiments, each delivered dose comprises about 60 to about 125 μg albuterol and about 25 to about 100 μg budesonide. In some embodiments, each delivered dose comprises about 80 to about 100 μg albuterol and about 30 to about 90 μg budesonide. In some embodiments, each delivered dose comprises about 90 μg albuterol and about 40 μg budesonide. In some embodiments, each delivered dose comprises about 90 μg albuterol and about 80 μg budesonide. In some embodiments, each delivered dose comprises about 45 μg albuterol and about 20 or about 40 μg budesonide. In some embodiments, each metered dose comprises one actuation by the metered dose inhaler.

In some embodiments, the subject is a human subject 12 years of age or older, and the administering comprises delivering to the subject two metered doses from the MDI via inhalation, wherein each delivered dose comprises (a) about 90 μg albuterol; and (b) about 60 to about 100 μg budesonide, or about 70 to about 90 μg budesonide, or about 80 μg budesonide. In some embodiments, the subject is a human subject 12 years of age or older, and the administering comprises delivering to the subject two metered doses from the MDI, wherein each delivered dose comprises (a) about 60 to about 130 μg albuterol, or about 70 to about 120 μg albuterol, or about 80 to about 100 μg albuterol, or about 90 μg albuterol; and (b) about 80 μg budesonide. In some embodiments, a total of (a) about 180 μg albuterol; and (b) about 120 to about 200 μg budesonide, or about 140 to about 180 μg budesonide, or about 160 μg budesonide are delivered via the two metered doses. In some embodiments, a total of (a) about 120 to about 250 μg albuterol, or about 140 to about 230 μg albuterol, or about 150 to about 200 μg albuterol, or about 180 μg albuterol; and (b) about 160 μg budesonide are delivered via the two metered doses. In some embodiments, the subject is not administered greater than 6 metered doses in a 24 hour period.

In some embodiments, the subject is a human subject from 4 to 11 years of age, and the administering comprises delivering to the subject two metered doses from the MDI via inhalation, wherein each delivered dose comprises (a) about 90 μg albuterol; and (b) about 20 to about 60 μg budesonide, or about 30 to about 50 μg budesonide, or about 40 μg budesonide. In some embodiments, the subject is a human subject from 4 to 11 years of age, and the administering comprises delivering to the subject two metered doses from the MDI, wherein each delivered dose comprises (a) about 60 to about 130 μg albuterol, or about 70 to about 120 μg albuterol, or about 80 to about 100 μg albuterol, or about 90 μg albuterol; and (b) about 40 μg budesonide. In some embodiments, a total of (a) about 180 μg albuterol and (b) about 40 to about 120 μg budesonide, or about 60 to about 100 μg budesonide, or about 80 μg budesonide are delivered via the two metered doses. In some embodiments, a total of (a) about 120 to about 250 μg albuterol, or about 140 to about 230 μg albuterol, or about 150 to about 200 μg albuterol, or about 180 μg albuterol; and (b) about 80 μg budesonide are delivered via the two metered doses. In some embodiments, the subject is not administered greater than 6 metered doses in a 24 hour period.

In some embodiments, about 110 to about 280 μg albuterol is delivered. In some embodiments, about 120 to about 250 μg albuterol is delivered. In some embodiments, about 140 to about 230 μg albuterol is delivered. In some embodiments, about 150 to about 200 μg albuterol is delivered. In some embodiments, about 150, about 155, about 160, about 165, about 170, about 175, about 180, about 185, about 190, about 195, or about 200 μg albuterol is delivered. In some embodiments, about 180 μg albuterol is delivered. In some embodiments, the administering comprises delivering two metered doses from the MDI, wherein each delivered dose comprises about 50 to about 150 μg, or about 60 to about 130 μg, or about 70 to about 120 μg, or about 80 to about 100 μg, or about 90 μg albuterol.

In some embodiments, about 40 to about 250 μg budesonide is delivered. In some embodiments, about 50 to about 200 μg budesonide is delivered. In some embodiments, about 75 to about 175 μg budesonide is delivered. In some embodiments, about 80 to about 160 μg budesonide is delivered. In some embodiments, about 40 to about 120 μg budesonide is delivered. In some embodiments, about 60 to about 100 μg budesonide is delivered. In some embodiments, about 120 to about 200 μg budesonide is delivered. In some embodiments, about 140 to about 180 μg is delivered. In some embodiments, about 60, about 70, about 80, about 90, about 100, about 110, about 120, about 130, about 140, about 150, about 160, about 170, about 180, about 190, or about 200 μg budesonide is delivered. In some embodiments, about 80 μg budesonide is delivered. In some embodiments, about 160 μg budesonide is delivered. In some embodiments, the administering comprises delivering two metered doses from the MDI, wherein each delivered dose comprises about 20 to about 130 μg, or about 30 to about 100 μg, or about 35 to about 90 μg, or about 40 to about 80 μg, or about 20 to about 60 μg, or about 30 to about 50 μg, or about 60 to about 100 μg, or about 70 to about 90 μg budesonide. In some embodiments, each delivered dose comprises about 40 μg budesonide. In some embodiments, each delivered dose comprises about 80 μg budesonide.

### Composition

In some embodiments, the disclosure provides a composition comprising: (a) a plurality of active agent particles comprising albuterol and budesonide; (b) a suspension medium; and (c) a plurality of respirable suspending particles, wherein the active agent particles of albuterol and budesonide associate with the plurality of respirable suspending particles. In some embodiments, the disclosure provides a composition comprising: (a) albuterol and budesonide in the form of particles as described herein; (b) a suspension medium comprising an HFO propellant and/or an HFC propellant; and (c) a plurality of respirable suspending particles, wherein the particles of albuterol and budesonide associate with the plurality of respirable suspending particles. In some embodiments, the plurality of active agent particles comprise a first plurality of particles of albuterol and a second plurality of particles of budesonide. That is, the plurality of albuterol particles and the plurality of budesonide particles are separate and different species of particles. In some embodiments, the composition is a co-suspension composition wherein the plurality of active agent particles, i.e., the plurality of albuterol particles and the plurality of budesonide particles, and the plurality of respirable suspending particles are co-suspended in the suspension medium to form the co-suspension. In some embodiments, the composition is for use in the treatment of asthma in a subject in need thereof.

In some embodiments, the albuterol and the budesonide are in the composition at a weight ratio of about 1:1 to about 5:1. In some embodiments, the albuterol and the budesonide are in the composition at a weight ratio of about 1:1 to about 4:1. In some embodiments, the albuterol and the budesonide are in the composition at a weight ratio of about 1:1 to about 3:1. In some embodiments, the albuterol and the budesonide are in the composition at a ratio of about weight 1.1:1 to about 2.8:1. the albuterol and the budesonide are in the composition at a weight ratio of about 1.1:1 to about 2.5:1. In some embodiments, the albuterol and the budesonide are in the composition at a weight ratio of about 1.1:1 or about 2.3:1. In some embodiments, the albuterol and the budesonide are in the composition at a weight ratio of about 1.125:1 or about 2.25:1. In some embodiments, the albuterol and the budesonide are in the composition at a weight ratio of about 1.125:1. In some embodiments, the albuterol and the budesonide are in the composition at a weight ratio of about 2.25:1.

In some embodiments, the composition delivers about 50 to about 150 μg albuterol and about 10 to about 130 μg budesonide per one or two metered doses when administered via a metered dose inhaler. In some embodiments, the composition delivers about 75 to about 100 μg albuterol and about 30 to about 90 μg budesonide per one or two metered doses when administered via a metered dose inhaler. In some embodiments, the composition delivers about 90 μg albuterol and about 40 or about 80 μg budesonide per one or two metered doses when administered via a metered dose inhaler. In some embodiments, the composition delivers about 45 μg albuterol and about 20 or about 40 μg budesonide per one or two metered doses when administered via a metered dose inhaler. Delivery of the compositions provided herein, e.g., via metered dose inhalers, is further described herein.

### Active Agent Particles

In some embodiments, the albuterol and the budesonide in the composition are in the form of particles. In some embodiments, the composition comprises a first plurality of particles of albuterol and a second plurality of particles of budesonide. In some embodiments, the composition comprising albuterol and budesonide described herein is a co-suspension. As described herein, a co-suspension comprises two or more types of particles within a suspension medium, wherein one type of particle associates at least partially with one or more of the other particle types. In some embodiments, the co-suspension comprises a suspension medium comprising particles of albuterol and budesonide (also referred to herein as “active agent particles”), a propellant, and suspending particles. In some embodiments, the active agent particles associate with the suspending particles such that separation of the active agent particles from the suspending particles is substantially prevented, resulting in co-location of the active agent particles and the suspending particles within the suspension medium. Co-suspension compositions are further described, e.g., in WO 2010/138862.

In some embodiments, the active agent particles (e.g., particles of albuterol and particles of budesonide) are formed of a material capable of being dispersed and suspended within the suspension medium and are sized to facilitate delivery of respirable particles from the co-suspension. In some embodiments, the active agent particles are provided as a micronized material wherein at least 90% of the active agent particles by volume exhibit an optical diameter of about 7 μm or less, or about 6 μm or less, or about 5 μm or less, or about 4 μm or less, or about 3 μm or less. In some embodiments, the active agent particles are provided as a micronized material wherein at least 90% of the active agent particles by volume exhibit an optical diameter of about 1 μm to about 7 μm, about 2 μm to about 5μ, or about 2 μm to about 3 μm. In some embodiments, the active agent particles are provided as a micronized material wherein at least 50% of the active agent particle material by volume exhibits an optical diameter of about 4 μm or less, or about 3 μm or less, or about 2 μm or less, or about 1.5 μm or less or about 1 μm or less, or about 1 μm to about 4 μm, or about 1 μm to about 3 μm, or about 1 μm to about 2 μm, or about 1.3 μm to about 1.9 μm.

The active agent particles (e.g., particles of albuterol and particles of budesonide) may be formed entirely of active agents (e.g., albuterol and budesonide, separately or in combination) or formulated to include one or more active agents in combination with one or more excipients or adjuvants. In some embodiments, the active agent particles comprise a first plurality of active agent particles and a second plurality of active agent particles. In some embodiments, the first plurality of active agent particles comprises albuterol and the second plurality of active agent particles comprises budesonide. In some embodiments, the first plurality of active agent particles comprise crystalline forms of albuterol. In some embodiments, the second plurality of active agent particles comprise amorphous forms of budesonide. In some embodiments, the first plurality of active agent particles comprise amorphous forms of albuterol. In some embodiments, the second plurality of active agent particles comprise crystalline forms of budesonide. In some embodiments, the active agent particles comprise both crystalline and amorphous forms of albuterol and budesonide. In some embodiments, one of albuterol and budesonide is provided in the crystalline form, and the other is provided in the amorphous form. Where the active agent particles described herein include the active agents in combination with one or more excipients or adjuvants, the excipients and adjuvants can be selected based on the chemical and physical properties of the active agent used. Moreover, suitable excipients for formulation of active agent particles include those described herein in association with the suspending particles. In some embodiments, the active agent particles are formulated with one or more of the lipid, phospholipid, carbohydrate, amino acid, organic salt, peptide, protein, alditols, synthetic or natural polymer, or surfactant materials as described, for example, in association with the suspending particles.

Any suitable process may be employed to achieve micronized active agent material, i.e., particles of albuterol and budesonide, for inclusion in the compositions described herein. A variety of processes may be used to create active agent particles suitable for use in the co-suspension formulations described herein, including, but not limited to, micronization by milling or grinding processes, crystallization or recrystallization processes, and processes using precipitation from supercritical or near-supercritical solvents, spray drying, spray freeze drying, or lyophilization. Suitable methods for obtaining micronized active agent particles include, for example, U.S. Pat. Nos. 6,063,138; 5,858,410; 5,851,453; 5,833,891; 5,707,634; and WO 2007/009164.

Where appropriate, the active agents (i.e., albuterol and budesonide) provided in the composition may be used in the form of salts (e.g., alkali metal or amine salts or as acid addition salts) or as esters, solvates (hydrates), derivatives, or a free base thereof. Additionally, the active agents (i.e., albuterol and budesonide) may be in any crystalline form or isomeric form or mixture of isomeric forms, for example, as pure enantiomers, a mixture of enantiomers, as racemates or as mixtures thereof. The appropriate form of the active agents for inclusion in the composition described herein may be selected by one of ordinary skill in the art. In some embodiments, the albuterol in the composition is albuterol sulfate. In some embodiments, the albuterol in the composition is crystalline albuterol sulfate particles. In some embodiments, the albuterol in the composition is micronized albuterol sulfate particles. In some embodiments, the albuterol in the composition is micronized crystalline albuterol sulfate particles. The structure of an exemplary albuterol sulfate compound is provided herein. In some embodiments, the budesonide in the composition comprises a racemic mixture of (22R) and (22S) stereoisomers of budesonide, as described herein. The structure of budesonide is provided herein. In some embodiments, the budesonide in the composition is micronized budesonide particles. In some embodiments, the albuterol in the composition comprises crystalline albuterol sulfate particles, and the budesonide in the composition comprises micronized budesonide particles. In some embodiments, the albuterol in the composition comprises micronized albuterol sulfate particles, and the budesonide in the composition comprises micronized budesonide particles.

### Suspending Particles

The suspending particles included in the co-suspension compositions described herein facilitate stabilization and delivery of the active agent, i.e., albuterol and budesonide, included in the compositions. Though various forms of suspending particles may be used, the suspending particles are typically formed from pharmacologically inert material that is acceptable for inhalation and is substantially insoluble in the propellant selected. Generally, the majority of suspending particles are sized within a respirable range. The terms “suspending particles” and “respirable suspending particles” are therefore used interchangeably throughout the present disclosure. In some embodiments, the mass median aerodynamic diameter (MMAD) of the suspending particles does not exceed about 10 μm but is not lower than about 500 nm. In some embodiments, the MMAD of the suspending particles is about 750 nm to about 5 μm. In some embodiments, the MMAD of the suspending particles is about 1 μm to about 3 μm. In some embodiments, the MMAD of the suspending particles is 1 μm to 10 μm.

In order to achieve respirable suspending particles within the MMAD ranges described, the suspending particles typically exhibit a volume median optical diameter of about 0.2 μm and about 50 μm. In some embodiments, the suspending particles exhibit a volume median optical diameter that does not exceed about 25 μm. In some embodiments, the suspending particles exhibit a volume median optical diameter of about 0.5 μm to about 15 μm, or about 1.5 μm to about 10 μm, or about 2 μm to about 5 μm.

The concentration of suspending particles included in the composition comprising albuterol and budesonide described herein can be adjusted, depending on, for example, the amount of active agent particles and suspension medium used. In some embodiments, the suspending particles are included in the suspension medium at a concentration of about 1 mg/mL to about 30 mg/mL, about 1 mg/mL to about 15 mg/mL, about 3 mg/mL to about 10 mg/mL, 5 mg/mL to about 8 mg/mL, or about 6 mg/mL. In some embodiments, the suspending particles are included in the suspension medium at a concentration of up to about 30 mg/mL. In some embodiments, the suspending particles are included in the suspension medium at a concentration of up to about 25 mg/mL.

The relative amounts of suspending particles to active agent particles (i.e., particles of albuterol and budesonide) are selected to achieve a co-suspension as described herein. A co-suspension composition may be achieved where the amount of suspending particles, as measured by mass, exceeds that of the active agent particles. In some embodiments, the ratio of the total mass of the suspending particles to the total mass of active agent particles is about 3:1 to about 15:1, or about 2:1 to about 8:1. In some embodiments, the ratio of the total mass of the suspending particles to the total mass of active agent particles is above about 1, such as up to about 1.5, up to about 5, up to about 10, up to about 15, up to about 17, up to about 20, up to about 30, up to about 40, up to about 50, up to about 60, up to about 75, up to about 100, up to about 150, and up to about 200, depending on the nature of the suspending particles and active agent particles used. In some embodiments, the ratio of the total mass of the suspending particles to the total mass of active agent particles is about 1.5:1 to about 200:1. In some embodiments, the ratio of the total mass of the suspending particles to the total mass of the active agent particles is about 10 to about 200, about 60 to about 200, about 15 to about 60, about 15 to about 170, about 15 to about 60, about 16, about 60, or about 170.

In some embodiments, the suspending particles comprise an excipient selected from a lipid, a phospholipid, a nonionic detergent, a polymer, a surfactant, a carbohydrate, an amino acid, an organic salt, a peptide, a protein, an alditol, or combinations thereof. In some embodiments, the suspending particles increase storage stability of the active agents, i.e., albuterol and budesonide. In some embodiments, the suspending particles include pharmaceutically acceptable glass stabilization excipients having a Tg of at least 55° C., at least 75° C., or at least 100° C. Exemplary suspending particles are provided, e.g., in WO 2010/138862; US RE 37,872; U.S. Pat. Nos. 5,928,469; 6,258,341; and 6,309,671. As used herein, Tg means glass transition temperature, i.e., the temperature range where the polymer substrate changes from a rigid glassy material to a soft (not melted) material, and is usually measured in terms of the stiffness, or modulus.

In some embodiments, the suspending particles comprise a phospholipid. In some embodiments, the phospholipid has a gel to liquid crystal phase transition of greater than about 40° C. Exemplary phospholipids include relatively long chain (i.e., C₁₆-C₂₂) saturated lipids, e.g., phosphatidylcholines having acyl chain lengths of 16 or 18 carbons (palmitoyl and stearoyl). Exemplary phospholipids include phosphoglycerides such as dipalmitoylphosphatidylcholine, disteroylphosphatidylcholine, diarachidoylphosphatidylcholine, dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, and long-chain saturated phosphatidylinositols. Additional excipients are described in WO 96/32149; U.S. Pat. Nos. 6,358,530; 6,372,258; and 6,518,239. In some embodiments, the suspending particles are phospholipid particles comprising 1,2-Distearoyl-sn-glycero-3-phosphocholine (DSPC).

In some embodiments, the suspending particles are perforated microstructures comprising DSPC and calcium chloride. Perforated microstructures are described in, e.g., WO 2010/138862 and refer to suspending particles which include a structural matrix that exhibits, defines, or comprises voids, pores, defects, hollows, spaces, interstitial spaces, apertures, perforations, or holes that allow the surrounding suspension medium to permeate, fill, or pervade the microstructure, such as those materials and preparations described in U.S. Pat. Nos. 6,309,623; 8,815,258; 9,463,161; and US 2011/0135737. In some embodiments, the suspending particles comprise perforated microstructures. Where perforated microstructures are used as suspending particles in the compositions described herein, they may include at least one of the following: lipids, phospholipids, nonionic detergents, nonionic block copolymers, ionic surfactants, biocompatible fluorinated surfactants and combinations thereof, particularly those approved for pulmonary use. Exemplary surfactants that may be used in the preparation of perforated microstructures include poloxamer 188, poloxamer 407 and poloxamer 338. Other exemplary surfactants include oleic acid or its alkali salts. In some embodiments, the perforated microstructures include greater than about 10% w/w surfactant.

### Propellant

In some embodiments, the composition comprising albuterol and budesonide, e.g., in the form of particles as described herein, further comprise a suspension medium, wherein the particles of albuterol and budesonide are in the suspension medium, and wherein the suspension medium further comprises a pharmaceutically acceptable propellant. In some embodiments, the suspension medium is a pharmaceutically acceptable propellant. In some embodiments, the suspension medium is a propellant of pharmaceutical grade (1E)-1,3,3,3-Tetrafluoro-1-propene (HFO-1234ze(E)). In general, suitable propellants are propellant gases that can be liquefied under pressure at room temperature, and upon inhalation or topical use, are safe and toxicologically innocuous. In some embodiments, the pharmaceutically acceptable propellant comprises a hydrofluoroalkane (HFA), a hydrofluoroolefins (HFO), a hydrofluorocarbon (HFC), a perfluorinated compound (PFC), a chlorofluorocarbon (CFC), or combinations thereof. Exemplary propellants are described in, e.g., WO 2010/138862.

In some embodiments, the composition described herein comprises an HFA propellant, such as, e.g., HFA-134a (1,1,1,2-tetrafluoroethane) and HFA-227 (1,1,1,2,3,3,3-Heptafluoropropane, also known as HFA-227ea). In some embodiments, the composition described herein comprises an HFO propellant and/or an HFC propellant. In some embodiments, the HFO propellant and/or HFC propellant is capable of providing a stable formulation that provides consistent dosing characteristics and is simple to formulate, i.e., without requiring modification by the addition of, for example, cosolvents, antisolvents, solubilizing agents, or adjuvants.

In some embodiments, the composition described herein comprises an HFO propellant. In some embodiments, the HFO propellant is pharmaceutical grade (1E)-1,3,3,3-tetrafluoropropene (HFO-1234ze(E)). The term “pharmaceutical grade propellant,” as used herein, indicates a propellant that is in compliance with the GMP regulations for use in humans. In some embodiments, the pharmaceutical grade HFO-1234ze(E) has a purity of at least about 99.90%.

In some embodiments, the composition described herein comprises an HFC propellant. In some embodiments, the HFC propellant is pharmaceutical grade 1,1-Difluoroethane (HFC-152a, also known as HFA-152a). In some embodiments, the pharmaceutical grade HFC-152a has a purity of at least about 99.90%.

In some embodiments, the disclosure provides for pharmaceutical compositions comprising albuterol and budesonide in the form of particles, a suspension medium comprising a hydrofluoroolefins (HFO) propellant, such as pharmaceutical grade HFO-1234ze(E) or a hydrofluorocarbon propellant (HFC), such as pharmaceutical grade HFC-152a, and a plurality of suspending particles, wherein the particles of albuterol and budesonide associate with the plurality of respirable suspending particles. In some embodiments, the disclosure provides for pharmaceutical compositions deliverable from a metered dose inhaler, the pharmaceutical composition comprising: a plurality of budesonide particles; a plurality of albuterol particles; a plurality of phospholipid particles; and a propellant of pharmaceutical grade (1E)-1,3,3,3-Tetrafluoro-1-propene (HFO-1234ze(E)) having a purity of at least about 99.90%; wherein the budesonide particles, the albuterol particles and the phospholipid particles are co-suspended in the propellant to form a co-suspension. In some embodiments of the co-suspension, the budesonide particles comprise micronized budesonide. In some embodiments of the co-suspension, the albuterol particles comprise micronized crystalline albuterol sulfate. In some embodiments, the composition comprises about 1 mg/mL to 30 mg/mL in the suspension medium, or about 5 mg/mL to about 25 mg/mL in the suspension medium. In some embodiments, the pharmaceutical composition comprises albuterol having a particle size of about 1 μm to about 7 μm. In some embodiments, the disclosure provides for an MDI canister comprising the pharmaceutical compositions described herein.

In some embodiments, a pharmaceutical composition, which comprises a plurality of budesonide particles; a plurality of albuterol particles; a plurality of phospholipid particles; and a propellant of pharmaceutical grade HFO-1234ze(E), exhibits similar or comparable bioavailability of the active agents (i.e., budesonide and albuterol) compared to a reference pharmaceutical composition, which comprises a plurality of budesonide particles; a plurality of albuterol particles; a plurality of phospholipid particles; and a propellant of pharmaceutical grade HFA-134a. In some embodiment, a pharmaceutical composition, which comprises a plurality of budesonide particles; a plurality of albuterol particles; a plurality of phospholipid particles; and a propellant of pharmaceutical grade HFO-1234ze(E), is bioequivalent to a reference pharmaceutical composition, which comprises a plurality of budesonide particles; a plurality of albuterol particles; a plurality of phospholipid particles; and a propellant of pharmaceutical grade HFA-134a. As used herein, a “reference pharmaceutical composition” means an alternative pharmaceutical composition which contains the same active agent particles (i.e., the budesonide particles and the albuterol particles) and the same suspending particles at the same respective concentrations as the pharmaceutical composition comprising the propellant of pharmaceutical grade HFO-1234ze(E). For example, a pharmaceutical composition and the reference pharmaceutical composition comprise the same active agent particles (i.e., the budesonide particles and the albuterol particles) and the same phospholipids particles at the same respective concentrations, but the reference pharmaceutical composition comprises a propellant of pharmaceutical grade HFA-134a, while the pharmaceutical composition comprises a propellant of pharmaceutical grade HFO-1234ze(E). In one embodiment, similar or comparable bioavailability or bioequivalence can be shown, wherein a ratio of the geometric mean of logarithmic transformed Cₘₐₓ, AUC_{inf} or AUCₗₐₛₜ for the two compositions (e.g., the pharmaceutical composition comprising pharmaceutical grade HFO-1234ze(E) and the reference pharmaceutical composition comprising pharmaceutical grade HFA-134a) is about 0.80 to about 1.25 with or without the 90% confidence interval (CI) limits.

Compositions formulated according to the present disclosure can inhibit degradation of the active agents, i.e., albuterol and budesonide, included therein. For example, in some embodiments, the compositions described herein inhibit one or more of flocculation, aggregation and Ostwald ripening of the active agent(s) included in the compositions, i.e., albuterol and budesonide. The stability provided by the compositions described herein allows the compositions to be dispensed in a manner that achieves desirable delivered dose uniformity throughout emptying of an MDI canister (“DDU”), even where the active agent to be delivered is highly potent and the delivered dose of the active agent is selected from, for example, less than one of 100 μg, 80 μg, 40 μg, 20 μg, 10 μg, 9 μg, 8 μg, 7 μg, 6 μg, 5 μg, 4 μg, 3 μg, 2 μg, 1 μg, 0.5 μg, and 0.1 μg per actuation of the MDI. In some embodiments, compositions described herein can achieve a DDU of ±30%, or better, for each of the active agents included in the composition, i.e., albuterol and budesonide. In some embodiments, compositions described herein achieve a DDU of ±25%, or better, for each of the active agents included in the composition, i.e., albuterol and budesonide. In some embodiments, compositions described herein achieve a DDU of ±20%, or better, ±15%, or better, or ±10%, or better, for each of the active agents included in the composition, i.e., albuterol and budesonide.

Moreover, compositions according to the present description serve to substantially preserve fine particle fraction (FPF) and fine particle dose (FPD) performance throughout emptying of an MDI canister, even after being subjected to accelerated degradation conditions. For instance, compositions according to the present description maintain as much as 80%, 85%, 90%, 95%, or more, of the original FPF and FPD performance. Compositions described herein provide the added benefit of achieving such performance while being formulated using non-CFC and non-HFA propellants. In some embodiments, the compositions described herein achieve desired one or more of a targeted DDU, FPF, and FPD performance while being formulated with suspension medium including only one or more HFO propellant and/or HFC propellant and without the need to modify the characteristics of the HFO propellant and/or HFC propellant, such as by the addition of, for example, one or more cosolvent, antisolvent, solubilizing agent, adjuvant or other propellant modifying material.

The stability and physical characteristics of the compositions described herein support several methods. In some embodiments, a method of formulating a pharmaceutical composition for respiratory delivery of an active agent is provided herein. The method involves the steps of providing a suspension medium comprising an HFO propellant and/or an HFC propellant described herein, albuterol and budesonide particles, and suspending particles as described herein, and combining such constituents to form a composition wherein the albuterol and budesonide particles associate with the suspending particles such that a co-suspension as described herein is formed. In some embodiments, the association of the albuterol and budesonide particles and the suspending particles is such that they do not separate due to their different buoyancies in the propellant, e.g., HFO and/or HFC propellant described herein.

The entire contents of all publications, patents, and patent applications referenced herein are hereby incorporated herein by reference.

The specific examples included herein are for illustrative purposes only and are not to be considered as limiting to this disclosure. Moreover, the compositions, systems and methods provided herein have been described in relation to certain embodiments thereof, and many details have been set forth for purposes of illustration. It will be apparent to those skilled in the art that the disclosure is susceptible to additional embodiments and that certain of the details described herein may be varied without departing from the basic principles of the disclosure. Any active agents and reagents used in the following examples are either commercially available or, with the benefit of the descriptions provided herein, can be prepared according to standard literature procedures by those skilled in the art.

### EXAMPLES

### Example 1. Phase 3 Clinical Trial—Albuterol and Budesonide Fixed Dose Combination (FDC) vs. Albuterol Alone

A global, Phase 3, randomized, double-blind, parallel-group, event-driven trial was conducted in patients with uncontrolled moderate-to-severe asthma receiving inhaled glucocorticoid maintenance medication, which was maintained throughout the trial. Adults and adolescents were randomized to all 3 arms: as-needed albuterol-budesonide 180/160-μg or 180/80-μg (delivered dose; 2 actuations of 90/80-μg and 90/40-μg, respectively) fixed-dose combination (FDC) or albuterol 180-μg (2 actuations of 90-μg). Children 4-11 years were randomized to only the lower albuterol-budesonide dose or albuterol 180-μg. Primary efficacy endpoint was time-to-first severe asthma exacerbation; secondary endpoints included annualized severe exacerbation rate and total systemic glucocorticoid exposure.

### Methods

### Patients

Symptomatic patients with asthma aged 4 years and older who had ≥1 severe asthma exacerbation in the previous 12 months (defined, for exacerbation history and primary endpoint, as a deterioration of asthma [worsening or new onset of symptoms] leading to ≥1 of the following: ≥3 consecutive days' treatment with systemic glucocorticoids to treat worsening symptoms of asthma [a single depot injection was considered equivalent to a 3-day burst]; an emergency department (ED) or urgent care visit for <24 hours requiring systemic glucocorticoid treatment as above; or an in-patient hospitalization for ≥24 hours due to asthma) were recruited. Additional inclusion criteria were forced expiratory volume in the first second (FEV₁)≥40 to <90% of predicted normal value for adults, without an upper limit for patients 4-17 years, and demonstrated in-clinic reversibility of FEV₁≥12% and an Asthma Control Questionnaire (ACQ-5) score of ≥1.5 at Visit 2 (Day 1 of double-blind study medication), indicating not well-controlled asthma.²⁵ Patients continued their maintenance medications throughout the trial. Major exclusion criteria included chronic obstructive pulmonary disease or other significant lung disease, systemic glucocorticoid use within 3 months before screening, and use of biologic treatments within 3 months or 5 half-lives before screening. Patients were receiving medium-to-high dose inhaled glucocorticoids or low-to-high dose inhaled glucocorticoids-long-acting beta₂-agonists (LABA), as defined by GINA,¹ with or without another controller, for ≥3 months with stable dosing for ≥4 weeks prior to Visit 1.

### Trial Design

The clinical trial was a global, Phase 3, randomized, double-blind, parallel-group, event-driven trial with 24-week minimum duration conducted at 295 sites. The trial continued until ≥570 severe exacerbation events were reported.

Adults and adolescents were randomized to all 3 trial arms (1:1:1 ratio): delivered doses of albuterol-budesonide 180/160-μg or albuterol-budesonide 180/80-μg, or albuterol 180-μg via pressurized metered dose inhaler (pMDI), administered as two as-needed actuations of albuterol-budesonide 90/80-μg, 90/40-μg, and albuterol 90-μg, respectively. Children 4-11 years were only randomized to the lower dose of albuterol-budesonide or albuterol 180-μg due to potential concerns of higher doses of inhaled glucocorticoids in this younger population. Patients were instructed on, and site staff confirmed, proper MDI technique. Patients were instructed to use study medication as needed in response to symptoms and could use study medication prior to exercise. Rescue use was limited to only study medication throughout the trial; additional fast-acting bronchodilators, including nebulizers, were prohibited for rescue use. Changes to maintenance therapy were allowed.

### Endpoints

The primary efficacy endpoint was time-to-first severe asthma exacerbation (i.e., the instantaneous risk of experiencing a first severe asthma exacerbation at any given time). Secondary efficacy endpoints were annualized severe exacerbation rate, total systemic glucocorticoid exposure for asthma over the treatment period, and responder analysis at Week 24 for the ACQ-5 (validated for ages≥6 years),²⁷ Asthma Quality of Life Questionnaire (AQLQ+12) for patients aged ≥12 years, and Pediatric Asthma Quality of Life Questionnaire (PAQLQ) for patients aged 7-11 years; patients aged 4-6 years completed the PAQLQ with the help of a caregiver.²⁸ ACQ-5 is scored on a scale from 0 to 6 (lower numbers indicating better asthma control; minimum clinically important difference [MCID], 0.5 points)²⁹ and AQLQ+12 and PAQLQ on a scale from 1 to 7 (higher scores indicating better asthma-related quality of life; MCID, 0.5 points).^{30,31} Responders were defined as patients with a decrease (ACQ-5) or increase (AQLQ+12/PAQLQ) of ≥0.5 from baseline score.

Safety endpoints (adverse events [AEs] and serious adverse events [SAEs]) were assessed from date of informed consent/assent through the safety follow-up.

### Statistical Analysis

For the primary endpoint, type I error was controlled for comparisons of each albuterol-budesonide dose with albuterol using the Hochberg procedure. Secondary endpoints were controlled using a hierarchical testing sequence for treatment comparisons albuterol-budesonide 180/160-μg versus albuterol and albuterol-budesonide 180/80-μg versus albuterol, grouped by secondary endpoint. A sample size of 1000 adults and adolescents per treatment group and 570 first severe exacerbation events provided 87% power to detect a 25% reduction in the risk of a severe exacerbation with albuterol-budesonide versus albuterol, assuming a 2-sided significance level of 5% and 0.22 exacerbation incidence for albuterol.²⁶ Additionally, the aim was to recruit 100 children aged 4-11 years in accordance with regulatory input.

The primary efficacy analyses were designed to investigate the benefit of albuterol-budesonide compared to albuterol in patients who did not change their maintenance treatment and did not discontinue randomized therapy. Data collected while on treatment prior to treatment discontinuation or change in maintenance therapy were used.²⁶ Supportive analyses were conducted using all available data including after any changes in maintenance therapy or premature discontinuation of as-needed study medication; i.e. a treatment policy strategy (de facto) was adopted for such events.

All randomized patients who received any amount of study drug, classified by the study medication they were randomized to, were included in the efficacy analyses comparing albuterol-budesonide 180/80-μg with albuterol. Children 4-11 years were excluded from the analyses of the 180/160-μg dose as they were not randomized to this arm. All patients receiving any amount of randomized study medication, and classified by the study medication they actually received, were included for all safety analyses.

The primary endpoint of time-to-first severe asthma exacerbation was analyzed using a Cox proportional hazards regression model adjusted for randomization stratification factors (age group, region) and the number of severe exacerbations in the 12 months before screening.

Annualized severe asthma exacerbation rate was analyzed using a negative binomial regression model, adjusting for age, region and number of severe exacerbations in the 12 months before screening and time at risk. Total systemic glucocorticoid exposure per patient was calculated as annualized total systemic glucocorticoid dose (mg/year). A Wilcoxon rank sum test was used with the objective of showing a difference between treatment groups in the location and spread of annualized total systemic glucocorticoid dose. Responder variables (ACQ-5 and AQLQ+12/PAQLQ) at Week 24 were analyzed using a logistic regression model adjusted for baseline values, randomization stratification factors (age group, region) and number of severe exacerbations in the 12 months prior to screening to compare treatment groups.

### Results

### Patients

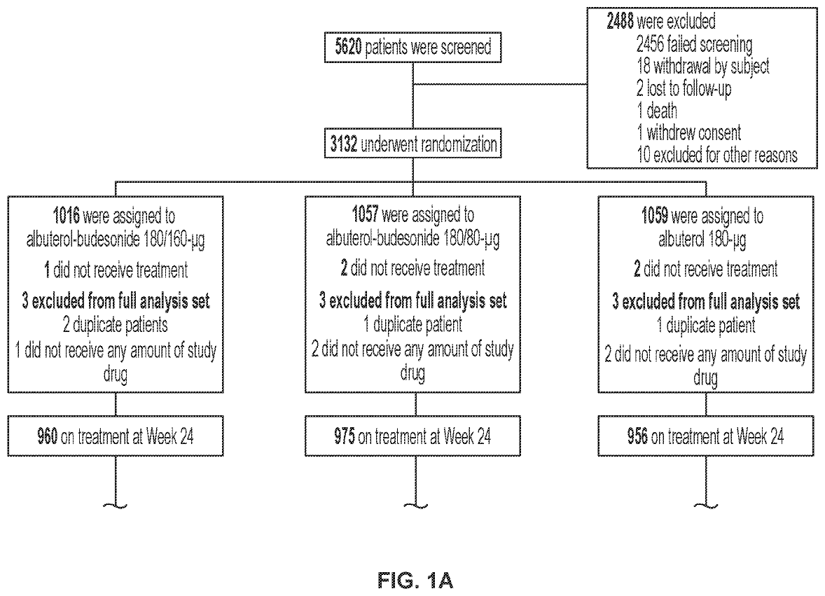

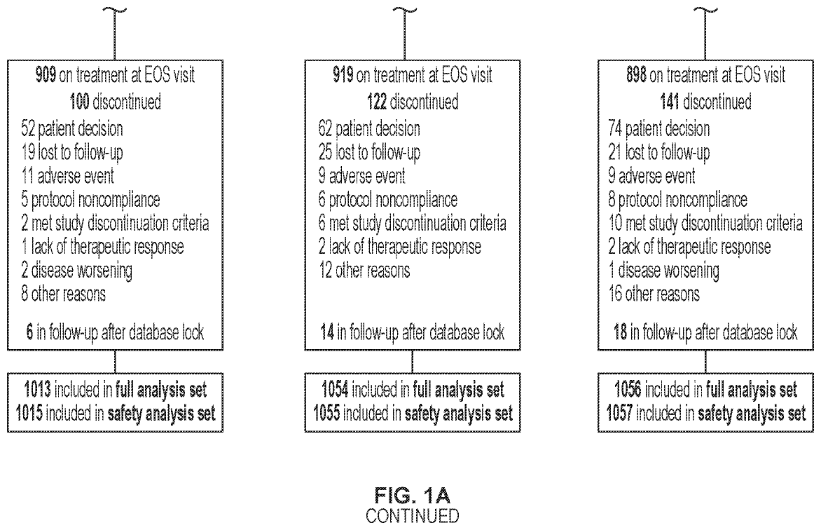

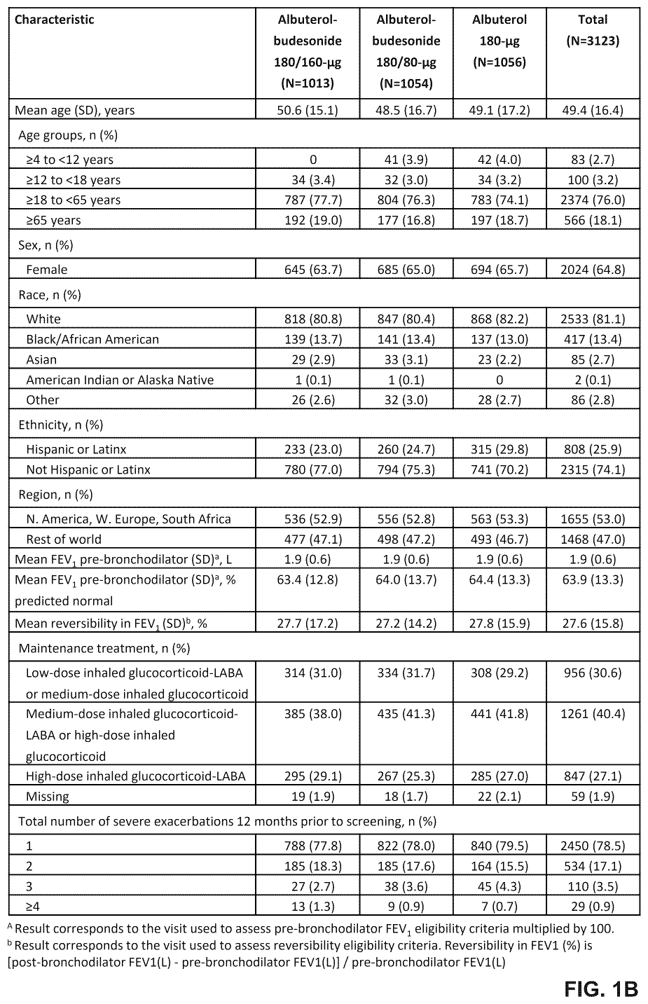

The trial enrolled 5620 patients, and 3132 were randomized (FIG. 1A). As indicated in FIG. 1A, patients on treatment at Week 24 refers to patients on their randomized treatment at Week 24, and those on treatment at the end of study (EOS) visit were defined as patients who attended the end of study visit on randomized treatment. The count of patients who discontinued included patients who discontinued the study during the extension phase (post Week 24). Data presented are from the primary database lock; 37 children and adolescents remained in the trial to complete 24 weeks.

Patient characteristics at screening are presented in FIG. 1B. Data shown in FIG. 1B are for all patients. Rescue use was limited to only study medication throughout the trial; additional fast-acting bronchodilators, including nebulizers, were prohibited. Additional controller medications were used by approximately 15% of patients, with approximately 10% on LTRA, 4% on LAMA and 2.2% on xanthines. Changes to maintenance therapy were allowed. At baseline, mean ACQ-5 score was 2.6 across treatment groups, indicating poorly controlled asthma. During the trial, patients reported that, on average, they had taken their maintenance medication on 74.7% of days (median 84.6%; standard deviation 25.6). Adherence was similar across treatment groups. Overall, 39 (1.2%) of patients changed maintenance therapy during the study.

### Primary Endpoint

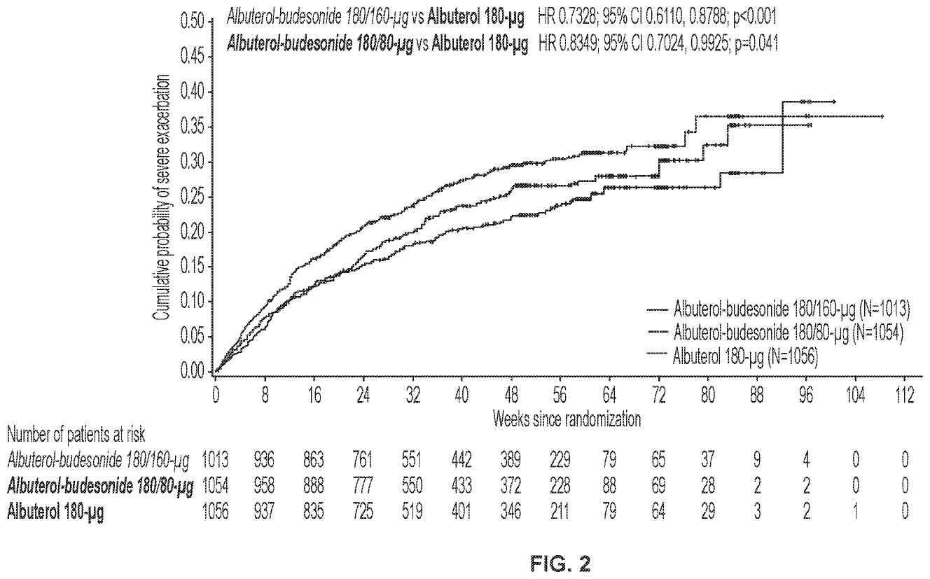

Both albuterol-budesonide doses demonstrated significant reductions in the risk of a severe exacerbation compared with albuterol (FIG. 2), as measured by time-to-first severe asthma exacerbation. Data shown in FIG. 2 are for all patients. The comparison for albuterol-budesonide 180/160-μg versus albuterol excludes children; therefore the albuterol population was n=1,014. For the comparison of albuterol-budesonide 180/160-μg versus albuterol, the number (%) of patients with a severe exacerbation was 207 (20.4%) in the albuterol-budesonide 180/160-μg group and 266 (26.2%) in the albuterol 180-μg group. For the comparison of albuterol-budesonide 180/80-μg versus albuterol, the number (%) of patients with a severe exacerbation was 241 (22.9%) in the albuterol-budesonide 180/80-μg group and 276 (26.1%) in the albuterol 180-μg group.

FIG. 2 shows that albuterol-budesonide reduced the risk of a severe exacerbation by 27% with the 180/160-μg dose (hazard ratio [HR] 0.7328; 95% confidence interval [CI] 0.6110, 0.8788; p<0.001), and 17% with the 180/80-μg dose (HR 0.8349; 95% CI 0.7024, 0.9925; p=0.041).

### Secondary Endpoints

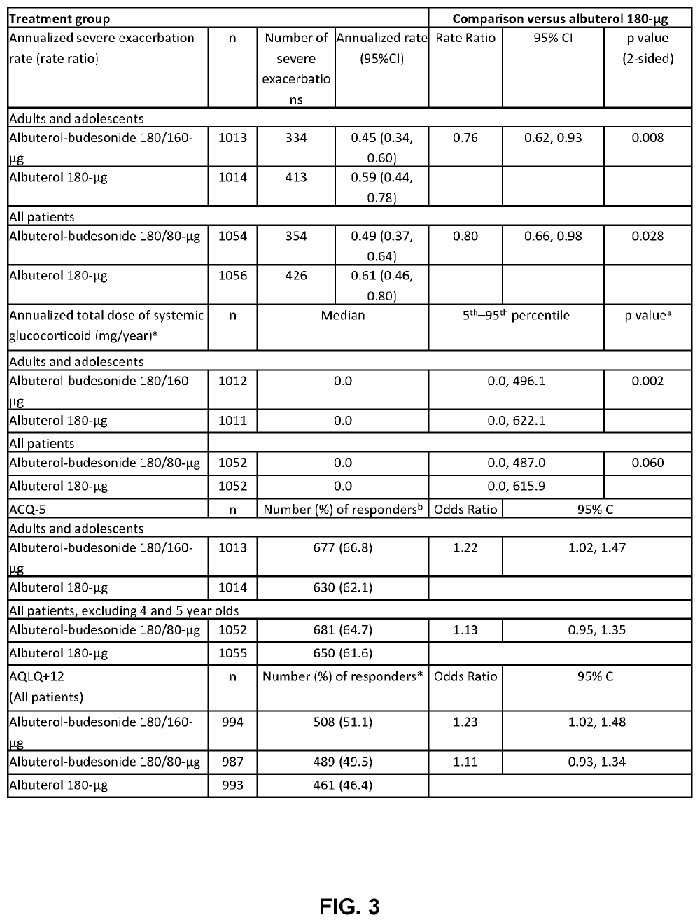

Secondary endpoint results are shown in FIG. 3. Data in FIG. 3 are presented for all patients, unless otherwise noted. The comparisons for albuterol-budesonide 180/160-μg versus albuterol excludes children and therefore the albuterol population was lower for these comparisons. The p-values for annualized total dose of systemic glucocorticoid were calculated via a Wilcoxon rank sum test separately for albuterol-budesonide 180/160-μg versus albuterol 180-μg and albuterol-budesonide 180/80-μg versus albuterol 180-μg. Responders in the Treatment Group were defined as patients with a decline (ACQ-5) or increase (AQLQ+12) from baseline score of at least 0.5. Multiple testing procedure was stopped after annualized total systemic glucocorticoids dose testing (hierarchical testing procedure); therefore 95% confidence intervals cannot be used to infer treatment effects. ACQ-5 is scored on a scale from 0 to 6 with lower numbers indicating better asthma control; minimum clinically important difference, 0.5 point. AQLQ(S)+12 is scored on a scale from 1 to 7 with higher scores indicating better asthma-related quality of life; minimum clinically important difference, 0.5 point.

Annualized severe exacerbation rate was 0.45 (95% CI 0.34, 0.60) for albuterol/budesonide 180/160-μg compared with 0.59 (95% CI 0.44, 0.78) for albuterol (rate ratio [RR] 0.76; 95% CI 0.62, 0.93; p=0.008), and 0.49 (95% CI 0.37, 0.64) for albuterol/budesonide 180/80-μg compared with 0.61 (95% CI 0.46, 0.80) for albuterol (RR 0.80; 95% CI 0.66, 0.98; p=0.028; FIG. 3). There were 9, 10, and 17 severe exacerbations requiring hospitalization in the albuterol-budesonide 180/160-μg, albuterol-budesonide 180/80-μg, and albuterol arms, respectively, and 49, 50, and 66 requiring ED or urgent care visits, respectively.

Annualized total systemic glucocorticoid exposure (prednisone equivalents) for albuterol-budesonide 180/160-μg (median 0.0 mg/year; 5^{th}-95^{th} percentile [0.0, 496.1]) compared with albuterol (median 0.0 mg/year; 5^{th}-95^{th} percentile [0.0, 622.1]) was significantly different (p=0.002; FIG. 3); this difference equates to a 33% reduction with budesonide 180/160-μg. The comparison between albuterol-budesonide 180/80-μg (median 0.0 mg/year; 5^{th}-95^{th} percentile [0.0, 487.0]) and albuterol (median 0.0 mg/year; 5^{th}-95^{th} percentile [0.0, 615.9]) was not significant (p=0.060; FIG. 3). Further statistical testing of secondary endpoints was not type I error controlled.

Results of the responder analyses for ACQ-5, AQLQ+12 and PAQLQ are also presented in FIG. 3.

### Study Medication Use

The overall pattern of as-needed study medication use was similar between treatment groups, with increased use around the time of an asthma deterioration. On a majority of trial days, patients reported using ≤2 inhalations (% mean trial days, albuterol-budesonide 180/160-μg: 53.7%; albuterol-budesonide 180/80-μg: 52.6%; albuterol 180-μg: 51.0%), and >8 inhalations on less than 2% of trial days. Average daily as-needed use was similar across treatment arms, with 2.6, 2.7, and 2.8 inhalations per day in patients treated with albuterol-budesonide 180/160-μg, albuterol-budesonide 180/80-μg, and albuterol 180-μg, respectively, equal to approximately 1.3, 1.4, and 1.4 doses of study medication per day.

### Safety Endpoints

The frequency of any AE was similar between randomized treatment groups: 46.2%, 47.1%, and 46.4% for albuterol-budesonide 180/160-μg, albuterol-budesonide 180/80-μg, and albuterol, respectively. The frequency of SAEs, including deaths, was 5.2%, 3.8%, and 4.5%, and the frequency of AEs leading to discontinuation of treatment was 1.0%, 0.9%, and 0.9% for albuterol-budesonide 180/160-μg, albuterol-budesonide 180/80-μg, and albuterol, respectively. There were 7 deaths: 4 in the albuterol-budesonide 180/160-μg group (2 cases of COVID-19, 1 case of elevated glucose, 1 cardiac arrest), 2 in the albuterol-budesonide 180/80-μg group (1 case of COVID-19, 1 case of lung metastasis with pneumothorax), and 1 in the albuterol group (COVID-19). No deaths were considered treatment-related.

The most common AEs included nasopharyngitis, headache, and upper respiratory tract infections. The frequency of inhaled glucocorticoid-associated adverse events was similar across treatment arms, ranging from 1.3% for albuterol to 2.0% for albuterol-budesonide 180/160-μg. The three most commonly reported treatment-related, investigator-assessed AEs across the albuterol-budesonide 180/160-μg, albuterol-budesonide 180/80-μg, and albuterol 180-μg treatment groups, respectively, were oral candidiasis (1.0%, 0.9%, 0.5%), dysphonia (0.4%, 0.6%, 0.4%), and oropharyngeal candidiasis (0.3%, 0.3%, 0.1%).

TABLE 1
Adverse events occurring in ≥2%
of patients in any treatment group

| | Albuterol- | Albuterol- | |
|---|---|---|---|
| | budesonide | budesonide | Albuterol- |
| | 180/160-μg | 180/80-μg | 180-μg |
| Preferred term | (N = 1015) | (N = 1055) | (N = 1057) |
| Patients with any AE, n (%) | 469 (46.2) | 497 (47.1) | 490 (46.4) |
| Nasopharyngitis | 76 (7.5) | 61 (5.8) | 54 (5.1) |
| Headache | 44 (4.3) | 50 (4.7) | 50 (4.7) |
| COVID-19 | 43 (4.2) | 52 (4.9) | 46 (4.4) |
| Upper respiratory tract | 26 (2.6) | 31 (2.9) | 26 (2.5) |
| infection | | | |
| Bronchitis | 25 (2.5) | 27 (2.6) | 28 (2.6) |
| Hypertension | 22 (2.2) | 27 (2.6) | 26 (2.5) |
| Asthma | 18 (1.8) | 20 (1.9) | 35 (3.3) |
| Back pain | 27 (2.7) | 23 (2.2) | 20 (1.9) |
| Influenza | 21 (2.1) | 23 (2.2) | 14 (1.3) |
| Sinusitis | 15 (1.5) | 17 (1.6) | 24 (2.3) |

### DISCUSSION

In patients with uncontrolled moderate-to-severe asthma receiving inhaled glucocorticoid-containing maintenance therapy, as-needed albuterol-budesonide FDC at doses of 180/160-μg (2 actuations of 90/80-μg) and 180/80-μg (2 actuations of 90/40-μg) significantly reduced severe exacerbation risk compared with as-needed albuterol. Significant reduction in annualized severe asthma exacerbation rate was observed with both albuterol-budesonide doses, and in total systemic glucocorticoid exposure with the 180/160-μg albuterol-budesonide dose compared with albuterol. Both doses of albuterol-budesonide demonstrated tolerability in line with the active components, with no safety concerns identified.

The pattern of rescue medication use was similar between treatment groups, both in terms of average overall use and in increased use around the time of an asthma deterioration. The average of <1.5 medication doses per day across treatment arms demonstrates that patients used albuterol-budesonide as they use albuterol. Unlike albuterol monotherapy, this FDC allows self-titration of inhaled glucocorticoid dose in response to the patient's own symptom-driven bronchodilator use during worsening asthma episodes.

In summary, both albuterol-budesonide FDC doses, used as needed on top of inhaled glucocorticoid-containing maintenance therapy, significantly reduced severe asthma exacerbation risk and rate versus albuterol in patients with uncontrolled moderate-to-severe asthma, with the albuterol-budesonide 180/160-μg dose also significantly reducing systemic glucocorticoid exposure.

### REFERENCES (INCORPORATED HEREIN IN THEIR ENTIRETY)

1. Global strategy for asthma management and prevention. 2021. (Accessed 29 Mar. 2022 at ginasthma.org/gina-reports/.)
2. O'Byrne P M, Jenkins C, Bateman E D. The paradoxes of asthma management: time for a new approach? Eur Respir J 2017; 50.
3. Reddel H K, FitzGerald J M, Bateman E D, et al. GINA 2019: a fundamental change in asthma management: Treatment of asthma with short-acting bronchodilators alone is no longer recommended for adults and adolescents. Eur Respir J 2019; 53.
4. 2020 Focused Updates to the Asthma Management Guidelines: A Report from the National Asthma Education and Prevention Program Coordinating Committee Expert Panel Working Group. 2020. (Accessed 29 Mar. 2022, at www.nhlbi.nih.gov/health-topics/all-publications-and-resources/2020-focused-updates-asthma-management-guidelines.)
5. Bloom C I, Cabrera C, Arnetorp S, et al. Asthma-Related Health Outcomes Associated with Short-Acting beta2-Agonist Inhaler Use: An Observational U K Study as Part of the SABINA Global Program. Adv Ther 2020; 37:4190-208.
6. Lugogo N, Gilbert I, Tkacz J, Gandhi H, Goshi N, Lanz M J. Real-world patterns and implications of short-acting beta2-agonist use in patients with asthma in the United States. Ann Allergy Asthma Immunol 2021; 126:681-9 e1.
7. Nwaru B I, Ekstrom M, Hasvold P, Wiklund F, Telg G, Janson C. Overuse of short-acting beta2-agonists in asthma is associated with increased risk of exacerbation and mortality: a nationwide cohort study of the global SABINA programme. Eur Respir J 2020; 55.
8. Bateman E D, Price D B, Wang H C, et al. Short-acting beta2-agonist prescriptions are associated with poor clinical outcomes of asthma: the multi-country, cross-sectional SABINA III study. Eur Respir J 2021.
9. Janson C, Menzies-Gow A, Nan C, et al. SABINA: An Overview of Short-Acting beta2-Agonist Use in Asthma in European Countries. Adv Ther 2020; 37:1124-35.
10. Quint J K, Arnetorp S, Kochs J W H, et al. Short-acting β2-agonist exposure and severe asthma exacerbations: SABINA findings from Europe and North America. J Allergy Clin Immunol Pract 2022.
11. O'Byrne P, Fabbri L M, Pavord I D, Papi A, Petruzzelli S, Lange P. Asthma progression and mortality: the role of inhaled corticosteroids. Eur Respir J 2019; 54.
12. Rabe K F, Atienza T, Magyar P, Larsson P, Jorup C, Lalloo U G. Effect of budesonide in combination with formoterol for reliever therapy in asthma exacerbations: a randomised controlled, double-blind study. Lancet 2006; 368:744-53.
13. Rogliani P, Ritondo B L, Ora J, Cazzola M, Calzetta L. SMART and as-needed therapies in mild-to-severe asthma: a network meta-analysis. Eur Respir J 2020; 56.
14. Sobieraj D M, Weeda E R, Nguyen E, et al. Association of Inhaled Corticosteroids and Long-Acting beta-Agonists as Controller and Quick Relief Therapy With Exacerbations and Symptom Control in Persistent Asthma: A Systematic Review and Meta-analysis. JAMA 2018; 319:1485-96.
15. Global strategy for asthma management and prevention. 2019. (Accessed 29 March, 2022 at ginasthma.org/gina-reports/.)
16. Reddel H K, Bacharier L B, Bateman E D, et al. Global Initiative for Asthma Strategy 2021: Executive Summary and Rationale for Key Changes. Am J Respir Crit Care Med 2022; 205:17-35.
17. Expert Panel Working Group of the National Heart L, Blood Institute a, coordinated National Asthma E, et al. 2020 Focused Updates to the Asthma Management Guidelines: A Report from the National Asthma Education and Prevention Program Coordinating Committee Expert Panel Working Group. J Allergy Clin Immunol 2020; 146:1217-70.
18. Bodzenta-Lukaszyk A, Dymek A, McAulay K, Mansikka H. Fluticasone/formoterol combination therapy is as effective as fluticasone/salmeterol in the treatment of asthma, but has a more rapid onset of action: an open-label, randomized study. BMC Pulm Med 2011; 11:28.
19. Bousquet J, Boulet L P, Peters M J, et al. Budesonide/formoterol for maintenance and relief in uncontrolled asthma vs. high-dose salmeterol/fluticasone. Respir Med 2007; 101:2437-46.
20. Kuna P, Peters M J, Manjra A I, et al. Effect of budesonide/formoterol maintenance and reliever therapy on asthma exacerbations. Int J Clin Pract 2007; 621:725-36.
21. O'Byrne P M, Bisgaard H, Godard P P, et al. Budesonide/formoterol combination therapy as both maintenance and reliever medication in asthma. Am J Respir Crit Care Med 2005; 171:129-36.
22. Scicchitano R, Aalbers R, Ukena D, et al. Efficacy and safety of budesonide/formoterol single inhaler therapy versus a higher dose of budesonide in moderate to severe asthma. Curr Med Res Opin 2004; 20:1403-18.
23. Albuterol sulfate HFA. (Accessed 29 Mar. 2022 at accessdata.fda.gov/drugsatfda_docs/label/2005/021457s001lbl.pdf.)
24. Usmani O S, Roche N, Jenkins M, Stjepanovic N, Mack P, De Backer W. Consistent Pulmonary Drug Delivery with Whole Lung Deposition Using the Aerosphere Inhaler: A Review of the Evidence. Int J Chron Obstruct Pulmon Dis 2021; 16:113-24.
25. Juniper E F, Bousquet J, Abetz L, Bateman E D, Committee G. Identifying ‘well-controlled’ and ‘not well-controlled’ asthma using the Asthma Control Questionnaire. Respir Med 2006; 100:616-21.
26. Chipps B E, Albers F C, Reilly L, Johnsson E, Cappelletti C, Papi A. Efficacy and safety of as-needed albuterol/budesonide versus albuterol in adults and children aged >/=4 years with moderate-to-severe asthma: rationale and design of the randomised, double-blind, active-controlled MANDALA study. BMJ Open Respir Res 2021; 8.
27. Juniper E F, O'Byrne P M, Guyatt G H, Ferrie P J, King D R. Development and validation of a questionnaire to measure asthma control. Eur Respir J 1999; 14:902-7.
28. Juniper E F, Buist A S, Cox F M, Ferrie P J, King D R. Validation of a standardized version of the Asthma Quality of Life Questionnaire. Chest 1999; 115:1265-70.
29. Bonini M, Di Paolo M, Bagnasco D, et al. Minimal clinically important difference for asthma endpoints: an expert consensus report. Eur Respir Rev 2020; 29.
30. Juniper E F, Guyatt G H, Feeny D H, Ferrie P J, Griffith L E, Townsend M. Measuring quality of life in children with asthma. Qual Life Res 1996; 5:35-46.
31. Juniper E F, Guyatt G H, Willan A, Griffith L E. Determining a minimal important change in a disease-specific Quality of Life Questionnaire. J Clin Epidemiol 1994; 47:81-7.
32. Papi A, Canonica G W, Maestrelli P, et al. Rescue use of beclomethasone and albuterol in a single inhaler for mild asthma. N Engl J Med 2007; 356:2040-52.
33. Israel E, Cardet J C, Carroll J K, et al. Reliever-Triggered Inhaled Glucocorticoid in Black and Latinx Adults with Asthma. N Engl J Med 2022.
34. Jenkins C R, Bateman E D, Sears M R, O'Byrne P M. What have we learnt about asthma control from trials of budesonide/formoterol as maintenance and reliever? Respirology 2020; 25:804-15.

### Example 2. Phase 3 Clinical Trial—Albuterol-Budesonide Combination Vs. Mono-Components

A Phase 3 clinical study evaluated contributions of the mono-components albuterol and budesonide to albuterol/budesonide combination efficacy in patients with mild-to-moderate asthma ≥4 years. Patients≥12 years were randomized 1:1:1:1:1 to four-times-daily albuterol/budesonide 180/160 or 180/80 μg, albuterol 180 μg, budesonide 160 μg or placebo for 12 weeks; patients 4-11 years were not included in this analysis set.

### Methods

In the study, 1001 patients with mild-to-moderate asthma were randomized 1:1:1:1:1 to ABD 180/160 μg or 180/80 μg, albuterol (A) 180 μg, budesonide (B) 160 μg, or placebo, all four times daily (QID), for 12 weeks. Dual-primary endpoints were change from baseline in forced expiratory volume in 1 second area under the curve from 0-6 hours (FEV₁, AUC₀₋₆ₕ) over 12 weeks and in trough FEV₁ at Week 12. Secondary endpoints included time to onset, as measured by 15% improvement in FEV₁ within 30 minutes on Day 1, and duration of effect, and Asthma Control Questionnaire-7 (ACQ-7) responder (≥0.5-point reduction from baseline) analysis at Week 12.

### Results

Of 1001 patients randomized, 989 were aged ≥12 years (mean age 48.9 years, 62.2% female). Change from baseline in FEV₁ AUC₀₋₆ₕ over 12 weeks was greater with albuterol/budesonide 180/160 μg (only dose tested) versus budesonide (least squares mean [LSM] difference 80.7 mL, 95% confidence interval [CI] 28.4-132.9; p=0.003; Table 2). Change in trough FEV₁ at Week 12 was greater with albuterol/budesonide 180/160 and 180/80 μg versus albuterol (LSM difference 132.8 mL [95% CI 63.6-201.9] and 120.8 mL [95% CI 51.5-190.1], respectively; both p<0.001; Table 2). Median time to onset and duration of effect on Day 1 (responders: 49.7%, 44.0% and 42.9%), was 7.5 and 7.0 minutes vs 9.5 minutes, and 186.9 and 191.4 minutes vs 168.2 minutes, respectively, for albuterol/budesonide 180/160 or 180/80 μg and albuterol. At Week 12, percentages of ACQ-7 responders were 66.5% and 65.5% vs 47.2% with albuterol/budesonide 180/160 or 180/80 μg vs albuterol (odds ratio [OR] 2.3 [95% CI 1.5-3.7] and OR 2.3 [95% CI 1.5-3.6]; both nominally significant), respectively. The safety profiles for both albuterol/budesonide doses were similar to the mono-components

### DISCUSSION

Both mono-components contributed to albuterol/budesonide efficacy, with the combinations demonstrating superior effects on lung function. At the time of initiating treatment, no difference was found in improvement in FEV₁ between albuterol/budesonide combination and albuterol 180 μg. With repeated use over 1-4 weeks, there was no difference in bronchodilation between albuterol 180 μg and placebo, whereas albuterol/budesonide combination was superior to albuterol 180 μg, with the difference increasing over time.

TABLE 2
Dual-primary endpoints in the full analysis set ≥12 years

| | | | | Comparison between | |
|---|---|---|---|---|---|
| | | | | groups | |
| | | | | Difference in | |
| Variable | Comparison | n | LSM | LSM (95% CI) | P value |
| Change from | Albuterol 180 μg vs placebo | 195 vs | 157.2 vs | 60.5 | 0.025 |
| baseline in | | 196 | 96.7 | (7.7, 113.4) | |
| FEV₁ AUC₀₋₆ | Albuterol/budesonide 180/160 | 197 vs | 258.6 vs | 161.9 | <0.001 |
| (mL) over | μg vs placebo | 196 | 96.7 | (109.4, 214.5) | |
| 12 weeks | *Albuterol/budesonide 180/160 | 197 vs | 258.6 vs | 80.7 | 0.003 |
| | μg vs budesonide 160 μg | 199 | 178.0 | (28.4, 132.9) | |
| Change from | Budesonide 160 μg vs placebo | 187 vs | 108.9 vs | 73.3 | 0.037 |
| baseline in | | 175 | 35.6 | (4.4, 142.2) | |
| trough FEV₁ | Albuterol/budesonide 180/160 | 186 vs | 135.5 vs | 99.9 | 0.005 |
| (mL) over 12 | μg vs placebo | 175 | 35.6 | (30.9, 168.8) | |
| weeks | *Albuterol/budesonide 180/160 | 186 vs | 135.5 vs | 132.8 | <0.001 |
| | μg vs albuterol 180 μg | 172 | 2.7 | (63.6, 201.9) | |
| | Albuterol/budesonide 180/80 | 184 vs | 123.5 vs | 87.9 | 0.013 |
| | μg vs placebo | 175 | 35.6 | (18.8, 156.9) | |
| | *Albuterol/budesonide 180/80 | 184 vs | 123.5 vs. | 120.8 | <0.001 |
| | μg vs albuterol 180 μg | 172 | 2.7 | (51.5, 190.1) | |

AUC_{0-6 h}, area under the curve from 0 to 6 hours; CI, confidence interval; FEV₁, forced expiratory volume in 1 second; LSM, least squares mean; n, number of patients in analysis.
*Primary endpoints.

1. A method of treating asthma in a subject at risk of asthma exacerbation, comprising administering as needed to the subject a composition comprising therapeutically effective amounts of albuterol and budesonide, wherein the composition is administered via a metered dose inhaler,
wherein the method reduces risk of severe asthma exacerbation by at least about 15% as compared to administration of a composition comprising a same dose of albuterol alone, as measured by time-to-first severe asthma exacerbation.

2. The method of claim 1, wherein the method reduces risk of severe asthma exacerbation by at least about 20% as compared to administration of a composition comprising a same dose of albuterol alone, as measured by time-to-first severe asthma exacerbation.

3. The method of claim 1 or 2, wherein the method reduces risk of severe asthma exacerbation by at least about 25% as compared to administration of a composition comprising a same dose of albuterol alone, as measured by time-to-first severe asthma exacerbation.

4. The method of any one of claims 1 to 3, wherein a hazard ratio of the method as compared to administration of a composition comprising a same dose of albuterol alone is less than about 0.9.

5. The method of any one of claims 1 to 4, wherein a hazard ratio of the method as compared to administration of a composition comprising a same dose of albuterol alone is less than about 0.8.

6. The method of any one of claims 1 to 5, wherein a hazard ratio of the method as compared to administration of a composition comprising a same dose of albuterol alone is less than about 0.7.

7. The method of any one of claims 1 to 6, wherein an annualized severe asthma exacerbation rate of the method is at least about 20% lower as compared to administration of a composition comprising a same dose of albuterol alone.

8. The method of any one of claims 1 to 7, wherein an annualized severe asthma exacerbation rate of the method is at least about 25% lower as compared to administration of a composition comprising a same dose of albuterol alone.

9. The method of any one of claims 1 to 8, wherein the method reduces the subject's number of severe asthma exacerbations requiring hospitalization by at least 30% as compared to administration of a composition comprising a same dose of albuterol alone.

10. The method of any one of claims 1 to 9, wherein the method reduces the subject's number of severe asthma exacerbations requiring emergency and/or urgent care by at least 20% as compared to administration of a composition comprising a same dose of albuterol alone.

11. The method of any one of claims 1 to 10, wherein the method reduces the subject's annualized total systemic corticosteroid dose by at least 30% as compared to administration of a composition comprising a same dose of albuterol alone.

12. The method of any one of claims 1 to 11, wherein the subject's forced expiratory volume in 1 second area under the curve from 0-6 hours (FEV₁ AUC₀₋₆) at 12 weeks following administration of the composition is greater than 110 mL.

13. The method of any one of claims 1 to 12, wherein the subject's FEV₁ AUC₀₋₆ at 12 weeks following administration of the composition is at least 10% higher as compared to a subject administered with a same dose of albuterol alone or a same dose of budesonide alone.

14. The method of any one of claims 1 to 13, wherein the subject's trough FEV₁ at 12 weeks following administration of the composition is greater than 90 mL.

15. The method of any one of claims 1 to 14, wherein the subject's trough FEV₁ at 12 weeks following administration of the composition is at least 10% higher as compared to a subject administered with a composition comprising a same dose of albuterol alone or a same dose of budesonide alone.

16. The method of any one of claims 1 to 15, wherein the administering comprises delivering one or more metered doses from the metered dose inhaler, wherein about 100 to about 300 μg albuterol and about 25 to about 250 μg budesonide are delivered.

17. The method of claim 16, wherein about 120 to about 250 μg albuterol is delivered.

18. The method of claim 16 or 17, wherein about 150 to about 200 μg albuterol is delivered.

19. The method of any one of claims 16 to 18, wherein about 180 μg albuterol is delivered.

20. The method of any one of claims 1 to 19, wherein about 50 to about 200 μg budesonide is delivered.

21. The method of any one of claims 1 to 20, wherein about 75 to about 175 μg budesonide is delivered.

22. The method of any one of claims 1 to 21, wherein about 80 or about 160 μg budesonide is delivered.

23. The method of any one of claims 1 to 22, wherein about 150 to about 200 μg albuterol and about 75 to about 175 μg budesonide are delivered.

24. The method of any one of claims 1 to 23, wherein about 180 μg albuterol and about 80 or about 160 μg budesonide are delivered.

25. The method of any one of claims 1 to 16, wherein the administering comprises delivering two metered doses from the metered dose inhaler, wherein each delivered dose comprises about 60 to about 125 μg albuterol and about 25 to about 100 μg budesonide.

26. The method of claim 25, wherein each delivered dose comprises about 90 μg albuterol and about 40 or about 80 μg budesonide.

27. The method of any one of claims 1 to 26, wherein the albuterol and the budesonide are in the composition at a weight ratio of about 1:1 to about 3:1.

28. The method of any one of claims 1 to 27, wherein the albuterol and the budesonide are in the composition at a weight ratio of about 1.1:1 to about 2.5:1.

29. The method of any one of claims 1 to 28, wherein the albuterol and the budesonide are in the composition at a weight ratio of about 1.125:1 or about 2.25:1.

30. The method of any one of claims 1 to 29, wherein the albuterol and the budesonide are in the form of particles, wherein at least 90% of the particles by volume comprise an optical diameter of about 1 μm to about 7 μm.

31. The method of claim 30, wherein the particles of albuterol comprise crystalline or micronized albuterol sulfate and the particles of budesonide comprise micronized budesonide.

32. The method of any one of claims 1 to 31, wherein the composition comprises a plurality of albuterol particles; a plurality of albuterol particles; a plurality of respirable suspending particles; and a suspension medium, wherein the plurality of albuterol particles, the plurality of budesonide particles, and the plurality of respirable suspending particles are co-suspended in the suspension medium to form a co-suspension.

33. The method of claim 32, wherein the suspension medium is a pharmaceutically acceptable propellant selected from a hydrofluoroalkane (HFA) propellant, a hydrofluoroolefins (HFO) propellant, a hydrofluorocarbon propellant (HFC), and combination thereof.

34. The method of claim 32 or 33, wherein the suspension medium is a propellant of pharmaceutical grade (1E)-1,3,3,3-Tetrafluoro-1-propene (HFO-1234ze(E)).

35. The method of any one of claims 32 to 34, wherein the respirable suspending particles comprise a volume median optical diameter of about 0.2 μm to about 50 μm.

36. The method of any one of claims 32 to 35, wherein the respirable suspending particles are about 1 mg/mL to about 30 mg/mL in the suspension medium.

37. The method of any one of claims 32 to 36, wherein the respirable suspending particles comprise a phospholipid.

38. The method of claim 37, wherein the phospholipid is 1,2-Distearoyl-sn-glycero-3-phosphocholine (DSPC).

39. The method of any one of claims 32 to 38, wherein a weight ratio of total mass of the respirable suspending particles to total mass of the albuterol and budesonide particles is about 1.5:1 to about 200:1.

40. A method of as-needed treatment or prevention of bronchoconstriction in a subject, and/or prevention of exacerbation in a subject with asthma, comprising administering as needed to the subject a composition comprising therapeutically effective amounts of albuterol and budesonide, wherein the composition is administered via a metered dose inhaler,
wherein the method reduces risk of severe asthma exacerbation by at least about 15% as compared to administration of a composition comprising a same dose of albuterol alone, as measured by time-to-first severe asthma exacerbation.

41. The method of claim 40, wherein the subject is a human subject 12 years of age or older.

42. The method of claim 41, wherein the administering comprises delivering to the subject two metered doses from the metered dose inhaler, wherein about 180 μg albuterol and about 160 μg budesonide are delivered.

43. The method of claim 40, wherein the subject is a human subject from 4 years to 11 years of age.

44. The method of claim 43, wherein the administering comprises delivering to the subject two metered doses from the metered dose inhaler, wherein about 180 μg albuterol and about 80 μg budesonide are delivered.

45. The method of any one of claims 40 to 44, wherein the subject is not administered greater than 6 metered doses in a 24 hour period.

46. A pharmaceutical composition deliverable from a metered dose inhaler comprising therapeutically effective amounts of albuterol and budesonide for use in treatment of asthma in a subject at risk of asthma exacerbation, wherein the treatment reduces risk of severe asthma exacerbation by at least about 15% as compared to administration of a composition comprising a same dose of albuterol alone, as measured by time-to-first severe asthma exacerbation.

47. A pharmaceutical composition deliverable from a metered dose inhaler comprising therapeutically effective amounts of albuterol and budesonide for use in as-needed treatment or prevention of bronchoconstriction in a subject and/or prevention of exacerbation in a subject with asthma, wherein the method reduces risk of severe asthma exacerbation by at least about 15% as compared to administration of a composition comprising a same dose of albuterol alone, as measured by time-to-first severe asthma exacerbation.

48. The pharmaceutical composition for use of claim 46 or 47, wherein the composition comprises a plurality of albuterol particles; a plurality of albuterol particles; a plurality of respirable suspending particles; and a suspension medium, wherein the plurality of albuterol particles, the plurality of budesonide particles, and the plurality of respirable suspending particles are co-suspended in the suspension medium to form a co-suspension.

49. The pharmaceutical composition for use of claim 48, wherein the suspension medium is a propellant of pharmaceutical grade (1E)-1,3,3,3-Tetrafluoro-1-propene (HFO-1234ze(E)).

50. The pharmaceutical composition for use of claim 48 or 49, wherein the respirable suspending particles comprise a phospholipid.

51. The pharmaceutical composition for use of claim 50, wherein the phospholipid is 1,2-Distearoyl-sn-glycero-3-phosphocholine (DSPC).

52. The pharmaceutical composition for use of any one of claims 46 to 51, wherein a hazard ratio of the treatment as compared to administration of a composition comprising a same dose of albuterol alone is less than about 0.9.

53. The pharmaceutical composition for use of any one of claims 46 to 52, wherein an annualized severe asthma exacerbation rate of the treatment is at least about 20% lower as compared to administration of a composition comprising a same dose of albuterol alone.
